(19) United States
(12) Patent Application Publication
(10) Pub. No.: US 2019/0323036 A1
(43) Pub. Date: Oct. 24, 2019

(54) METHOD TO BUILD FUNGAL PRODUCTION STRAINS USING AUTOMATED STEPS FOR GENETIC MANIPULATION AND STRAIN PURIFICATION

(71) Applicant: ZYMERGEN INC., EMERYVILLE, CA (US)

(72) Inventors: Kenneth S. BRUNO, Walnut Creek, CA (US); Patrick WESTFALL, Walnut Creek, CA (US); Edyta SZEWCZYK, Walnut Creek, CA (US); Kyle ROTHSCHILD-MANCINELLI, Woodside, CA (US)

(21) Appl. No.: 16/453,260

(22) Filed: Jun. 26, 2019

Related U.S. Application Data
(63) Continuation of application No. PCT/US2017/069086, filed on Dec. 29, 2017.
(60) Provisional application No. 62/441,040, filed on Dec. 30, 2016.

(51) Int. Cl. C12N 15/87; C12N 1/14; C12M 1/36; G01N 35/00; G01N 35/10; C12N 15/63; C12N 15/10

(52) U.S. Cl. CPC C12N 15/87; C12N 1/14; C12M 41/48; C12N 2510/00; G01N 35/10; C12N 15/635; C12N 15/1037; G01N 35/00029

(57) ABSTRACT

The present disclosure provides a high-throughput (HTP) microbial genomic engineering method and system for transforming, screening, and selecting filamentous fungal cells that utilizes automation. The method and system utilize HTP selection and counter-selection to purify homokaryotic transformed filamentous fungal cells. Furthermore, the present disclosure provides a method for producing and long-term storage of protoplasts derived from filamentous fungal cells.

### CROSS-REFERENCE TO RELATED APPLICATIONS

This application claims priority from U.S. Provisional Application Ser. No. 62/441,040, filed Dec. 30, 2016, which is herein incorporated by reference in its entirety for all purposes.

### FIELD

The present disclosure is directed to automated fungal genomic engineering. The disclosed automated genomic engineering platform entails the genetic manipulation of filamentous fungi to generate fungal production strains as well as facilitate purification thereof. The resultant fungal production strains are well-suited for growth in sub-merged cultures, e.g., for the large-scale production of products of interest (e.g., antibiotics, metabolites, proteins, etc.) for commercial applications.

### BACKGROUND

Eukaryotic cells are preferred organisms for the production of polypeptides and secondary metabolites. In fact, filamentous fungi are capable of expressing native and heterologous proteins to high levels, making them well-suited for the large-scale production of enzymes and other proteins for industrial, pharmaceutical, animal health and food and beverage applications. However, use of filamentous fungi for large-scale production of products of interest often requires genetic manipulation of said fungi as well as use of automated machinery and equipment and certain aspects of the filamentous fungal life cycle can make genetic manipulation and handling difficult.

For example, DNA introduced into a fungus integrates randomly within a genome, resulting in mostly random integrated DNA fragments, which quite often can be integrated as multiple tandem repeats (see for example Casqueiro et al., 1999, J. Bacteriol. 181:1181-1188). This uncontrolled “at random multiple integration” of an expression cassette can be a potentially detrimental process, which can lead to unwanted modification of the genome of the host.

Additionally, present transfection systems for filamentous fungi can be very laborious (see for review Fincham, 1989, Microbiol. Rev. 53:148-170) and relatively small scale in nature. This can involve protoplast formation, viscous liquid handling (i.e. polyethylene glycol solutions), one-by-one swirling of glass tubes and subsequent selective plating. Further, conditions for protoplasting can be difficult to determine and yields can often be quite low. Moreover, the protoplasts can contain multiple nuclei such that introduction of a desired genetic manipulation can lead to the formation of heterokaryotic protoplasts that can be difficult to separate from homokaryotic protoplasts.

Further, typical filamentous fungal cells, including those derived from protoplasts, grow as long fibers called hyphae that can form dense networks of hyphae called mycelium. These hyphae can contain multiple nuclei that can differ from one another in genotype. The hyphae can differentiate and form asexual spores that can be easily dispersed in the air. If the hyphae contain nuclei of different genotypes, the spores will also contain a mixture of nuclei. Due to this aspect of fungal growth, genetic manipulation inherently results in a mixed population that must be purified to homogeneity in order to assess any effect of the genetic changes made. Further, in an automated environment, the spores can cause contamination of equipment that could negatively impact the ability to purify strains and may contaminate any other work performed on the equipment.

To mitigate the aerial dispersal of spores, the filamentous fungi can be grown in submerged cultures. However, the mycelium formed by hyphal filamentous fungi growth in submerged cultures can affect the rheological properties of the broth. Generally, the higher the viscosity of the broth, the less uniform the distribution of oxygen and nutrients, and the more energy required to agitate the culture. In some cases, the viscosity of the broth due to hyphal filamentous fungal growth becomes sufficiently high to significantly interfere with the dissolution of oxygen and nutrients, thereby adversely affecting the growth of the fungi and ultimately the yield and productivity of any desired product of interest.

Thus, there is a great need in the art for new methods of engineering filamentous fungi, which do not suffer from the aforementioned drawbacks inherent with traditional strain building programs in fungi and greatly accelerate the process of discovering and consolidating beneficial mutations.

The current disclosure overcomes many of the challenges inherent in genetically manipulating filamentous fungi in an automated, high-throughput platform. The methods provided herein are designed to generate fungal production strains by incorporating genetic changes using automated co-transformation, or automated split marker design transformation, combined with automated screening of transformants thereby allowing exchange of genetic traits between two strains without going through a sexual cross. This disclosure also provides a procedure for generating large numbers of protoplasts and a means to store them for later use. Large batches of readily available competent cells can greatly facilitate automation.

### SUMMARY OF THE DISCLOSURE

In one aspect, provided herein is a method for producing a filamentous fungal strain, the method comprising: a.) providing a plurality of protoplasts, wherein the protoplasts were prepared from a culture of filamentous fungal cells; b). transforming the plurality of protoplasts with a first construct and a second construct, wherein the first construct comprises a first polynucleotide flanked on both sides by nucleotides homologous to a first locus in the genome of the protoplast and the second construct comprises a second polynucleotide flanked on both sides by nucleotides homologous to a second locus in the genome of the protoplast, wherein transformation results in integration of the first construct into the first locus and the second construct into the second locus by homologous recombination, wherein at least the second locus is a first selectable marker gene in the protoplast genome, and wherein the first polynucleotide comprises mutation and/or a genetic control element; c.) purifying homokaryotic transformants by performing selection and counter-selection; and d.) growing the purified transformants in media conducive to regeneration of the filamentous fungal cells. In another aspect, provided herein is a method for producing a filamentous fungal strain, the method comprising: a.) providing a plurality of protoplasts, wherein the protoplasts were prepared from a culture of filamentous fungal cells; b.) transforming the plurality of protoplasts with a first construct and a second construct, wherein the first construct comprises a first polynucleotide flanked by nucleotides homologous to a locus in the genome of the protoplast and the second construct comprises a second polynucleotide flanked by nucleotides homologous to the locus in the genome of the protoplast, wherein the first polynucleotide and second polynucleotides comprise complementary portions of a selectable marker, and wherein the first construct and/or the second construct further comprise a mutation or genetic control element, wherein transformation results in integration of the first and second polynucleotide and the mutation or genetic control element into the locus by homologous recombination; c.) purifying homokaryotic transformants by performing selection and counter-selection; and d.) growing the purified transformants in media conducive to regeneration of the filamentous fungal cells.

In some cases, each protoplast from the plurality of protoplasts is transformed with a single first construct from a plurality of first constructs and a single second construct from a plurality of second constructs, wherein the first polynucleotide in each first construct from the plurality of first constructs comprises a different mutation and/or genetic control element; and wherein the second polynucleotide in each second construct from the plurality of second constructs is identical. In some cases, the method further comprises repeating steps a-d to generate a library of filamentous fungal cells, wherein each filamentous fungal cell in the library comprises a first polynucleotide with a different mutation and/or genetic control element. In some cases, the first polynucleotide encodes a target filamentous fungal gene or a heterologous gene. In some cases, the mutation is a single nucleotide polymorphism. In some cases, the genetic control is a promoter sequence and/or a terminator sequence. In some cases, the plurality of protoplasts are distributed in wells of a microtiter plate. In some cases, steps a-d are performed in wells of a microtiter plate. In some cases, the microtiter plate is a 96 well, 384 well or 1536 well microtiter plate. In some cases, the filamentous fungal cells are selected from Achlya, Acremonium, Aspergillus, Aureobasidium, Bjerkandera, Ceriporiopsis, Cephalosporium, Chrysosporium, Cochliobolus, Corynascus, Cryphonectria, Cryptococcus, Coprinus, Coriolus, Diplodia, Endothis, Fusarium, Gibberella, Gliocladium, Humicola, Hypocrea, Myceliophthora (e.g., Myceliophthora thermophila), Mucor, Neurospora, Penicillium, Podospora, Phlebia, Piromyces, Pyricularia, Rhizomucor, Rhizopus, Schizophyllum, Scytalidium, Sporotrichum, Talaromyces, Thermoascus, Thielavia, Tramates, Tolypocladium, Trichoderma, Verticillium, Volvariella species or teleomorphs, or anamorphs, and synonyms or taxonomic equivalents thereof. In some cases, the filamentous fungal cells are Aspergillus niger. In some cases, the filamentous fungal cells possess a non-mycelium forming phenotype. In some cases, wherein the fungal cell possesses a non-functional non-homologous end joining (NHEJ) pathway. In some cases, the NHEJ pathway is made non-functional by exposing the cell to an antibody, a chemical inhibitor, a protein inhibitor, a physical inhibitor, a peptide inhibitor, or an anti-sense or RNAi molecule directed against a component of the NHEJ pathway. In some cases, the first locus is for the target filamentous fungal gene. In some cases, the first locus is for a second selectable marker gene in the protoplast genome. In some cases, the second selectable marker gene is selected from an auxotrophic marker gene, a colorimetric marker gene or a directional marker gene. In some cases, the first selectable marker gene is selected from an auxotrophic marker gene, a colorimetric marker gene or a directional marker gene. In some cases, the second polynucleotide is selected from an auxotrophic marker gene, a directional marker gene or an antibiotic resistance gene. In some cases, the colorimetric marker gene is an aygA gene. In some cases, the auxotrophic marker gene is selected from an argB gene, a trpC gene, a pyrG gene, or a met3 gene. In some cases, the directional marker gene is selected from an acetamidase (amdS) gene, a nitrate reductase gene (niaD), or a sulphate permease (Sut B) gene. In some cases, the antibiotic resistance gene is a ble gene, wherein the ble gene confers resistance to pheomycin. In some cases, the first selectable marker gene is an aygA gene and the second polynucleotide is a pyrG gene. In some cases, the first selectable marker gene is a met3 gene, the second selectable marker gene is an aygA gene and the second polynucleotide is a pyrG gene. In some cases, the plurality of protoplasts are prepared by removing cell walls from the filamentous fungal cells in the culture of filamentous fungal cells; isolating the plurality of protoplasts; and resuspending the isolated plurality of protoplasts in a mixture comprising dimethyl sulfoxide (DMSO), wherein the final concentration of DMSO is 7% v/v or less. In some cases, the mixture is stored at at least −20° C. or −80° C. prior to performing steps a-d. In some cases, the culture is at least 1 liter in volume. In some cases, the culture is grown for at least 12 hours prior to preparation of the protoplasts. In some cases, the fungal culture is grown under conditions whereby at least 70% of the protoplasts are smaller and contain fewer nuclei. In some cases, removing the cell walls is performed by enzymatic digestion. In some cases, the enzymatic digestion is performed with mixture of enzymes comprising a beta-glucanase and a polygalacturonase. In some cases, the method further comprises adding 40% v/v polyethylene glycol (PEG) to the mixture comprising DMSO prior to storing the protoplasts. In some cases, the PEG is added to a final concentration of 8% v/v or less. In some cases, steps a-d are automated.

In another aspect, provided herein is a method for preparing filamentous fungal cells for storage, the method comprising: preparing protoplasts from a fungal culture comprising filamentous fungal cells, wherein the preparing the protoplasts comprises removing cell walls from the filamentous fungal cells in the fungal culture; isolating the protoplasts; and resuspending the isolated protoplasts in a mixture comprising dimethyl sulfoxide (DMSO) at a final concentration of 7% v/v or less. In some cases, the mixture is stored at at least −20° C. or −80° C. In some cases, the fungal culture is at least 1 liter in volume. In some cases, the fungal culture is grown for at least 12 hours prior to preparation of the protoplasts. In some cases, the fungal culture is grown under conditions whereby at least 70% of the protoplasts are smaller and have fewer nuclei. In some cases, removing the cell walls is performed by enzymatic digestion. In some cases, the enzymatic digestion is performed with mixture of enzymes comprising a beta-glucanase and a polygalacturonase. In some cases, the method further comprises adding 40% v/v polyethylene glycol (PEG) to the mixture comprising DMSO prior to storing the protoplasts. In some cases, the PEG is added to a final concentration of 8% v/v or less. In some cases, the method further comprises distributing the protoplasts into microtiter plates prior to storing the protoplasts. In some cases, the filamentous fungal cells in the fungal culture possess a non-mycelium forming phenotype. In some cases, the filamentous fungal cells in the fungal culture are selected from Achlya, Acremonium, Aspergillus, Aureobasidium, Bjerkandera, Ceriporiopsis, Cephalosporium, Chrysosporium, Cochliobolus, Corynascus, Cryphonectria, Cryptococcus, Coprinus, Coriolus, Diplodia, Endothis, Fusarium, Gibberella, Gliocladium, Humicola, Hypocrea, Myceliophthora (e.g., Myceliophthora thermophila), Mucor, Neurospora, Penicillium, Podospora, Phlebia, Piromyces, Pyricularia, Rhizomucor, Rhizopus, Schizophyllum, Scytalidium, Sporotrichum, Talaromyces, Thermoascus, Thielavia, Tramates, Tolypocladium, Trichoderma, Verticillium, Volvariella species or teleomorphs, or anamorphs, and synonyms or taxonomic equivalents thereof. In some cases, the filamentous fungal cells in the fungal culture are Aspergillus niger or teleomorphs or anamorphs thereof.

In yet another aspect, provided herein is a system for generating a fungal production strain, the system comprising: one or more processors; and one or more memories operatively coupled to at least one of the one or more processors and having instructions stored thereon that, when executed by at least one of the one or more processors, cause the system to: a.) transform a plurality of protoplasts derived from culture of filamentous fungal cells with a first construct and a second construct, wherein the first construct comprises a first polynucleotide flanked on both sides by nucleotides homologous to a first locus in the genome of the protoplast and the second construct comprises a second polynucleotide flanked on both sides by nucleotides homologous to a second locus in the genome of the protoplast, wherein transformation results in integration of the first construct into the first locus and the second construct into the second locus by homologous recombination, wherein at least the second locus is a first selectable marker gene in the protoplast genome, and wherein the first polynucleotide comprises a mutation and/or a genetic control element; b.) purifying homokaryotic transformants by performing selection and counter-selection; and c.) growing the purified transformants in media conducive to regeneration of the filamentous fungal cells. In some cases, each protoplast from the plurality of protoplasts is transformed with a single first construct from a plurality of first constructs and a single second construct from a plurality of second constructs, wherein the first polynucleotide in each first construct from the plurality of first constructs comprises a different mutation and/or genetic control element; and wherein the second polynucleotide in each second construct from the plurality of second constructs is identical. In some cases, the system further comprises repeating steps a-c to generate a library of filamentous fungal cells, wherein each filamentous fungal cell in the library comprises a first polynucleotide with a different mutation and/or genetic control element. In some cases, the mutation is a single nucleotide polymorphism. In some cases, the genetic control is a promoter sequence and/or a terminator sequence. In some cases, steps a-c are performed in wells of a microtiter plate. In some cases, the microtiter plate is a 96 well, 384 well or 1536 well microtiter plate. In some cases, the filamentous fungal cells are selected from Achlya, Acremonium, Aspergillus, Aureobasidium, Bjerkandera, Ceriporiopsis, Cephalosporium, Chrysosporium, Cochliobolus, Corynascus, Cryphonectria, Cryptococcus, Coprinus, Coriolus, Endothis, Fusarium, Gibberella, Gliocladium, Humicola, Hypocrea, Myceliophthora (e.g., Myceliophthora thermophila), Mucor, Neurospora, Penicillium, Podospora, Phlebia, Piromyces, Pyricularia, Rhizomucor, Rhizopus, Schizophyllum, Scytalidium, Sporotrichum, Tataromyces, Thermoascus, Thielavia, Tramates, Tolypocladium, Trichoderma, Verticillium, Volvariella species or teleomorphs, or anamorphs, and synonyms or taxonomic equivalents thereof. In some cases, the filamentous fungal cells are Aspergillus niger. In some cases, the filamentous fungal cells possess a non-mycelium forming phenotype. In some cases, the fungal cell possesses a non-functional non-homologous end joining pathway. In some cases, the NHEJ pathway is made non-functional by exposing the cell to an antibody, a chemical inhibitor, a protein inhibitor, a physical inhibitor, a peptide inhibitor, or an anti-sense or RNAi molecule directed against a component of the NHEJ pathway. In some cases, the first locus is for the target filamentous fungal gene. In some cases, the first locus is for a second selectable marker gene in the protoplast genome. In some cases, the second selectable marker gene is selected from an auxotrophic marker gene, a colorimetric marker gene or a directional marker gene. In some cases, the first selectable marker gene is selected from an auxotrophic marker gene, a colorimetric marker gene or a directional marker gene. In some cases, the second polynucleotide is selected from an auxotrophic marker gene, a directional marker gene or an antibiotic resistance gene. In some cases, the colorimetric marker gene is an aygA gene. In some cases, the auxotrophic marker gene is selected from an argB gene, a trpC gene, a pyrG gene, or a met3 gene. In some cases, the directional marker gene is selected from an acetamidase (amdS) gene, a nitrate reductase gene (nlaD), or a sulphate permease (Sut B) gene. In some cases, the antibiotic resistance gene is a hie gene, wherein the ble gene confers resistance to pheomycin. In some cases, the first selectable marker gene is an aygA gene and the second polynucleotide is a pyrG gene. In some cases, the first selectable marker gene is a met3 gene, the second selectable marker gene is an aygA gene and the second polynucleotide is a pyrG gene. In some cases, the plurality of protoplasts are prepared by removing cell walls from the filamentous fungal cells in the culture of filamentous fungal cells; isolating the plurality of protoplasts; and resuspending the isolated plurality of protoplasts in a mixture comprising dimethyl sulfoxide (DMSO) at a final concentration of 7% v/v or less. In some cases, the mixture is stored at at least −20° C. or −80° C. prior to performing steps a-c. In some cases, the culture is at least 1 liter in volume. In some cases, the culture is grown for at least 12 hours prior to preparation of the protoplasts. In some cases, the fungal culture is grown under conditions whereby at least 70% of the protoplasts are smaller and have fewer nuclei. In some cases, removing the cell walls is performed by enzymatic digestion. In some cases, the enzymatic digestion is performed with mixture of enzymes comprising a beta-glucanase and a polygalacturonase. In some cases, the system further comprises adding 40% v/v polyethylene glycol (PEG) to the mixture comprising DMSO prior to storing the protoplasts. In some cases, the PEG is added to a final concentration of 8% v/v or less.

### BRIEF DESCRIPTION OF THE FIGURES

FIG. 1A depicts a general outline for the automated transformation, screening, and purification of homokaryotic protoplasts provided herein and described in Example 1.

FIG. 1B is a representation of how SNPs are targeted to a specific locus in filamentous fungi using a split marker system. The marker gene (pyrG in this example) is amplified into two components that are unable to compliment the mutation in the target strain without homologous recombination, which restores gene function. Flanking these fragments is a direct repeat of DNA that each of which contain the SNPs to be targeted to the locus. Non-repeat DNA sequence on each construct facilitates proper integration through native homologous recombination pathways. These constructs are placed into the target strains during step 2 of FIG. 1D.

FIG. 1C illustrates that the direct repeats flanking the marker gene are unstable and will result in marker removal through homologous recombination between the direct repeats. Marker removal is carried out using media containing counter selection of the marker represented in step 6 of FIG. 1D. This process is often called “loopout” or “looping out.”

FIG. 1D illustrates steps in the process of SNP swapping in filamentous fungi.

FIG. 1E illustrates steps in the process of screening the transformants for proper integration.

FIG. 2 depicts screening of A. niger mutant strains utilizing the argB marker by observing growth of A. niger mutant strains on minimal media with and without arginine following automated transformation and screening as described in Example 2.

FIG. 3 depicts screening of A. niger mutant strains utilizing the aygA colorimetric gene marker by observing growth of A. niger mutant strains on minimal media following automated transformation and screening as described in Example 3. Colonies derived from homokaryotic protoplasts were pure yellow in color and lacked black spores.

FIG. 4A-B depicts the results of A. niger transformation and validation according to the methods of the present disclosure. FIG. 4A—is a picture of a 96-well media plate of A. niger transformants. Transformed cultures comprise a mutation in the aygA, which causes the cells to appear lighter yellow instead of black (transformed wells are circled in white). FIG. 4B—depicts the results of next generation sequencing of transformed A. niger mutants. The X-axis represents the target DNA's sequence identity with the untransformed parent strain. The Y-axis represents the target DNA's sequence identity with the expected mutation. Data points towards the bottom right of the chart exhibit high similarity with the parent strain, and low similarity with the expected transformed sequences. Data points towards the top left of the chart exhibit high similarity to expected transformed sequences and low identity with parent strain. Data points in the middle likely represent heterokaryons with multiple nuclei.

FIG. 4C depicts the results of A. niger split marker design transformation and validation according to the methods of the present disclosure. The data was generated using next generation sequencing of transformed (via split marker) A. niger mutants. And is a distribution of the match to the mutation at the target vs match to parent at the target. Every sample in the top left corner of this graph are correct and have passed QC. The samples within the circle contain both the mutant and parent at the locus and may be processed again through steps 4 and 5 of FIG. 1D in order to generate isolates that may pass QC.

FIG. 5 depicts a SNP swap implementation in A. Niger. The left side of FIG. 5 illustrates the designed genetic edits for each SNP of the SNP swap. The figure further illustrates the cotransformation in which the pyrG gene is introduced into the locus for the aygA wild type gene. The right side of FIG. 5 shows two pictures of the 96-well media plates for screening the A. niger transformants. Light yellow colonies represent transformants in which the aygA gene has been successfully disrupted. The A. niger strain used to build the mutant strains depicted within FIG. 5 were strains with reduced NHEJ pathway activity.

FIG. 6 is a graphic representation of the next generation sequencing data from a SNPSWP campaign. In this example, 31 loci were targeted using constructs designed as presented in FIG. 1B. Here 1264 total isolates were screened by sequencing each amplicon populations from all individual samples. This data set contained over one million sequenced amplicons. There were 119 samples that passed all QC requirements. Quality control includes checking for the presence of parental mutation at the loci and all of the amplicons from the well must match the target DNA across the entire amplicon. Samples in red (+symbol) are correct, samples that are blue (dot symbol) may contain both the parent and the mutation.

FIG. 7 depicts one embodiment of the automated system of the present disclosure. The present disclosure teaches use of automated robotic systems with various modules capable of cloning, transforming, culturing, screening and/or sequencing host organisms.

FIG. 8 diagrams an embodiment of a computer system, according to embodiments of the present disclosure.

### DETAILED DESCRIPTION

### Definitions

While the following terms are believed to be well understood by one of ordinary skill in the art, the following definitions are set forth to facilitate explanation of the presently disclosed subject matter.

The term “a” or “an” refers to one or more of that entity, i.e. can refer to a plural referents. As such, the terms “a” or “an”, “one or more” and “at least one” are used interchangeably herein. In addition, reference to “an element” by the indefinite article “a” or “an” does not exclude the possibility that more than one of the elements is present, unless the context clearly requires that there is one and only one of the elements.

As used herein the terms “cellular organism” “microorganism” or “microbe” should be taken broadly. These terms are used interchangeably and include, but are not limited to, the two prokaryotic domains, Bacteria and Archaea, as well as certain eukaryotic fungi and protists. In some embodiments, the disclosure refers to the “microorganisms” or “cellular organisms” or “microbes” of lists/tables and figures present in the disclosure. This characterization can refer to not only the identified taxonomic genera of the tables and figures, but also the identified taxonomic species, as well as the various novel and newly identified or designed strains of any organism in said tables or figures. The same characterization holds true for the recitation of these terms in other parts of the Specification, such as in the Examples.

The term “prokaryotes” is art recognized and refers to cells which contain no nucleus or other cell organelles. The prokaryotes are generally classified in one of two domains, the Bacteria and the Archaea. The definitive difference between organisms of the Archaea and Bacteria domains is based on fundamental differences in the nucleotide base sequence in the 16S ribosomal RNA.

The term “Archaea” refers to a categorization of organisms of the division Mendosicutes, typically found in unusual environments and distinguished from the rest of the prokaryotes by several criteria, including the number of ribosomal proteins and the lack of muramic acid in cell walls. On the basis of ssrRNA analysis, the Archaea consist of two phylogenetically-distinct groups: Crenarchaeota and Euryarchaeota. On the basis of their physiology, the Archaea can be organized into three types: methanogens (prokaryotes that produce methane); extreme halophiles (prokaryotes that live at very high concentrations of salt (NaCl); and extreme (hyper) thermophilus (prokaryotes that live at very high temperatures). Besides the unifying archaeal features that distinguish them from Bacteria (i.e., no murein in cell wall, ester-linked membrane lipids, etc.), these prokaryotes exhibit unique structural or biochemical attributes which adapt them to their particular habitats. The Crenarchaeota consists mainly of hyperthermophilic sulfur-dependent prokaryotes and the Euryarchaeota contains the methanogens and extreme halophiles.

“Bacteria” or “eubacteria” refers to a domain of prokaryotic organisms. Bacteria include at least 11 distinct groups as follows: (1) Gram-positive (gram+) bacteria, of which there are two major subdivisions: (1) high G+C group (Actinomycetes, Mycobacteria, Micrococcus, others) (2) low G+C group (Bacillus, Clostridia, Lactobacillus, Staphylococci, Streptococci, Mycoplasmas); (2) Proteobacteria, e.g., Purple photo synthetic+non-photo synthetic Gram-negative bacteria (includes most “common” Gram-negative bacteria); (3) Cyanobacteria, e.g., oxygenic phototrophs; (4) Spirochetes and related species; (5) Planctomyces; (6) Bacteroides, Flavobacteria; (7) Chlamydia; (8) Green sulfur bacteria; (9) Green non-sulfur bacteria (also anaerobic phototrophs); (10) Radioresistant micrococci and relatives; (11) Thermotoga and Thermosipho thermophiles.

A “eukaryote” is any organism whose cells contain a nucleus and other organelles enclosed within membranes. Eukaryotes belong to the taxon Eukarya or Eukaryota. The defining feature that sets eukaryotic cells apart from prokaryotic cells (the aforementioned Bacteria and Archaea) is that they have membrane-bound organelles, especially the nucleus, which contains the genetic material, and is enclosed by the nuclear envelope.

The terms “genetically modified host cell,” “recombinant host cell,” and “recombinant strain” are used interchangeably herein and refer to host cells that have been genetically modified by the cloning and transformation methods of the present disclosure. Thus, the terms include a host cell (e.g., fungal cell, etc.) that has been genetically altered, modified, or engineered, such that it exhibits an altered, modified, or different genotype and/or phenotype (e.g., when the genetic modification affects coding nucleic acid sequences of the microorganism), as compared to the naturally-occurring or parental organism from which it was derived. It is understood that the terms refer not only to the particular recombinant host cell in question, but also to the progeny or potential progeny of such a host cell

The term “wild-type microorganism” or “wild-type strain” describes a cell that occurs in nature, i.e. a cell that has not been genetically modified.

The term “parent strain” or “parental strain” or “parent” may refer to a host cell from which mutant strains are derived. Accordingly, the “parent strain” or “parental strain” is a host cell or cell whose genome is perturbed by any manner known in the art and/or provided herein to generate one or more mutant strains. The “parent strain” or “parental strain” may or may not have a genome identical to that of a wild-type strain.

The term “genetically engineered” may refer to any manipulation of a host cell's genome (e.g. by insertion, deletion, mutation, or replacement of nucleic acids).

The term “control” or “control host cell” refers to an appropriate comparator host cell for determining the effect of a genetic modification or experimental treatment. In some embodiments, the control host cell is a wild type cell. In other embodiments, a control host cell is genetically identical to the genetically modified host cell, save for the genetic modification(s) differentiating the treatment host cell. In some embodiments, the present disclosure teaches the use of parent strains as control host cells. In other embodiments, a host cell may be a genetically identical cell that lacks a specific SNP being tested in the treatment host cell.

As used herein, the term “allele(s)” means any of one or more alternative forms of a gene, all of which alleles relate to at least one trait or characteristic. In a diploid cell, the two alleles of a given gene occupy corresponding loci on a pair of homologous chromosomes. Since the present disclosure, in embodiments, relates to QTLs, i.e. genomic regions that may comprise one or more genes or regulatory sequences, it is in some instances more accurate to refer to “haplotype” (i.e. an allele of a chromosomal segment) instead of “allele”, however, in those instances, the term “allele” should be understood to comprise the term “haplotype”.

As used herein, the term “locus” (loci plural) means a specific place or places or a site on a chromosome where for example a gene or genetic marker is found.

A “recombination” or “recombination event” as used herein refers to a chromosomal crossing over or independent assortment. The term “recombinant” refers to an organism having a new genetic makeup arising as a result of a recombination event.

As used herein, the term “phenotype” refers to the observable characteristics of an individual cell, cell culture, organism, or group of organisms which results from the interaction between that individual's genetic makeup (i.e., genotype) and the environment.

As used herein, the term “nucleic acid” refers to a polymeric form of nucleotides of any length, either ribonucleotides or deoxyribonucleotides, or analogs thereof. This term refers to the primary structure of the molecule, and thus includes double- and single-stranded DNA, as well as double- and single-stranded RNA. It also includes modified nucleic acids such as methylated and/or capped nucleic acids, nucleic acids containing modified bases, backbone modifications, and the like. The terms “nucleic acid” and “nucleotide sequence” are used interchangeably.

As used herein, the term “gene” refers to any segment of DNA associated with a biological function. Thus, genes include, but are not limited to, coding sequences and/or the regulatory sequences required for their expression. Genes can also include non-expressed DNA segments that, for example, form recognition sequences for other proteins. Genes can be obtained from a variety of sources, including cloning from a source of interest or synthesizing from known or predicted sequence information, and may include sequences designed to have desired parameters.

As used herein, the term “homologous” or “homologue” or “ortholog” is known in the art and refers to related sequences that share a common ancestor or family member and are determined based on the degree of sequence identity. The terms “homology,” “homologous,” “substantially similar” and “corresponding substantially” are used interchangeably herein. They refer to nucleic acid fragments wherein changes in one or more nucleotide bases do not affect the ability of the nucleic acid fragment to mediate gene expression or produce a certain phenotype. These terms also refer to modifications of the nucleic acid fragments of the instant disclosure such as deletion or insertion of one or more nucleotides that do not substantially alter the functional properties of the resulting nucleic acid fragment relative to the initial, unmodified fragment. It is therefore understood, as those skilled in the art will appreciate, that the disclosure encompasses more than the specific exemplary sequences. These terms describe the relationship between a gene found in one species, subspecies, variety, cultivar or strain and the corresponding or equivalent gene in another species, subspecies, variety, cultivar or strain. For purposes of this disclosure homologous sequences are compared. “Homologous sequences” or “homologues” or “orthologs” are thought, believed, or known to be functionally related. A functional relationship may be indicated in any one of a number of ways, including, but not limited to: (a) degree of sequence identity and/or (b) the same or similar biological function. Preferably, both (a) and (b) are indicated. Homology can be determined using software programs readily available in the art, such as those discussed in Current Protocols in Molecular Biology (F. M. Ausubel et al., eds., 1987) Supplement 30, section 7.718, Table 7.71. Some alignment programs are MacVector (Oxford Molecular Ltd, Oxford, U.K.), ALIGN Plus (Scientific and Educational Software, Pennsylvania) and AlignX (Vector NTI, Invitrogen, Carlsbad, Calif.). Another alignment program is Sequencher (Gene Codes, Ann Arbor, Mich.), using default parameters.

As used herein, the term “endogenous” or “endogenous gene,” refers to the naturally occurring gene, in the location in which it is naturally found within the host cell genome. In the context of the present disclosure, operably linking a heterologous promoter to an endogenous gene means genetically inserting a heterologous promoter sequence in front of an existing gene, in the location where that gene is naturally present. An endogenous gene as described herein can include alleles of naturally occurring genes that have been mutated according to any of the methods of the present disclosure.

As used herein, the term “exogenous” is used interchangeably with the term “heterologous,” and refers to a substance coming from some source other than its native source. For example, the terms “exogenous protein,” or “exogenous gene” refer to a protein or gene from a non-native source or location, and that have been artificially supplied to a biological system.

As used herein, the term “nucleotide change” refers to, e.g., nucleotide substitution, deletion, and/or insertion, as is well understood in the art. For example, mutations contain alterations that produce silent substitutions, additions, or deletions, but do not alter the properties or activities of the encoded protein or how the proteins are made.

As used herein, the term “at least a portion” or “fragment” of a nucleic acid or polypeptide means a portion having the minimal size characteristics of such sequences, or any larger fragment of the full length molecule, up to and including the full length molecule. A fragment of a polynucleotide of the disclosure may encode a biologically active portion of a genetic regulatory element. A biologically active portion of a genetic regulatory element can be prepared by isolating a portion of one of the polynucleotides of the disclosure that comprises the genetic regulatory element and assessing activity as described herein. Similarly, a portion of a polypeptide may be 4 amino acids, 5 amino acids, 6 amino acids, 7 amino acids, and so on, going up to the full length polypeptide. The length of the portion to be used will depend on the particular application. A portion of a nucleic acid useful as a hybridization probe may be as short as 12 nucleotides; in some embodiments, it is 20 nucleotides. A portion of a polypeptide useful as an epitope may be as short as 4 amino acids. A portion of a polypeptide that performs the function of the full-length polypeptide would generally be longer than 4 amino acids.

Variant polynucleotides also encompass sequences derived from a mutagenic and recombinogenic procedure such as DNA shuffling. Strategies for such DNA shuffling are known in the art. See, for example, Stemmer (1994) PNAS 91:10747-10751; Stemmer (1994) Nature 370:389-391; Crameri et al. (1997) Nature Biotech. 15:436-438; Moore et al. (1997) J. Mol. Biol. 272:336-347; Zhang et al. (1997) PNAS 94:4504-4509; Crameri et al. (1998) Nature 391:288-291; and U.S. Pat. Nos. 5,605,793 and 5,837,458.

For PCR amplifications of the polynucleotides disclosed herein, oligonucleotide primers can be designed for use in PCR reactions to amplify corresponding DNA sequences from cDNA or genomic DNA extracted from any organism of interest. Methods for designing PCR primers and PCR cloning are generally known in the art and are disclosed in Sambrook et al. (2001) Molecular Cloning: A Laboratory Manual (3^{rd} ed., Cold Spring Harbor Laboratory Press, Plainview, N.Y.). See also Innis et al., eds. (1990) PCR Protocols: A Guide to Methods and Applications (Academic Press, New York); Innis and Gelfand, eds. (1995) PCR Strategies (Academic Press, New York); and Innis and Gelfand, eds. (1999) PCR Methods Manual (Academic Press, New York). Known methods of PCR include, but are not limited to, methods using paired primers, nested primers, single specific primers, degenerate primers, gene-specific primers, vector-specific primers, partially-mismatched primers, and the like.

The term “primer” as used herein refers to an oligonucleotide which is capable of annealing to the amplification target allowing a DNA polymerase to attach, thereby serving as a point of initiation of DNA synthesis when placed under conditions in which synthesis of primer extension product is induced, i.e., in the presence of nucleotides and an agent for polymerization such as DNA polymerase and at a suitable temperature and pH. The (amplification) primer is preferably single stranded for maximum efficiency in amplification. Preferably, the primer is an oligodeoxyribonucleotide. The primer must be sufficiently long to prime the synthesis of extension products in the presence of the agent for polymerization. The exact lengths of the primers will depend on many factors, including temperature and composition (A/T vs. G/C content) of primer. A pair of bi-directional primers consists of one forward and one reverse primer as commonly used in the art of DNA amplification such as in PCR amplification.

The terms “stringency” or “stringent hybridization conditions” refer to hybridization conditions that affect the stability of hybrids, e.g., temperature, salt concentration, pH, formamide concentration and the like. These conditions are empirically optimized to maximize specific binding and minimize non-specific binding of primer or probe to its target nucleic acid sequence. The terms as used include reference to conditions under which a probe or primer will hybridize to its target sequence, to a detectably greater degree than other sequences (e.g. at least 2-fold over background). Stringent conditions are sequence dependent and will be different in different circumstances. Longer sequences hybridize specifically at higher temperatures. Generally, stringent conditions are selected to be about 5° C. lower than the thermal melting point (Tm) for the specific sequence at a defined ionic strength and pH. The Tm is the temperature (under defined ionic strength and pH) at which 50% of a complementary target sequence hybridizes to a perfectly matched probe or primer. Typically, stringent conditions will be those in which the salt concentration is less than about 1.0 M Na+ion, typically about 0.01 to 1.0 M Na+ion concentration (or other salts) at pH 7.0 to 8.3 and the temperature is at least about 30° C. for short probes or primers (e.g. 10 to 50 nucleotides) and at least about 60° C. for long probes or primers (e.g. greater than 50 nucleotides). Stringent conditions may also be achieved with the addition of destabilizing agents such as formamide. Exemplary low stringent conditions or “conditions of reduced stringency” include hybridization with a buffer solution of 30% formamide, 1 M NaCl, 1% SDS at 37° C. and a wash in 2×SSC at 40° C. Exemplary high stringency conditions include hybridization in 50% formamide, 1M NaCl, 1% SDS at 37° C., and a wash in 0.1×SSC at 60° C. Hybridization procedures are well known in the art and are described by e.g. Ausubel et al., 1998 and Sambrook et al., 2001. In some embodiments, stringent conditions are hybridization in 0.25 M Na2HPO4 buffer (pH 7.2) containing 1 mM Na2EDTA, 0.5-20% sodium dodecyl sulfate at 45° C., such as 0.5%, 1%, 2%, 3%, 4%, 5%, 6%, 7%, 8%, 9%, 10%, 11%, 12%, 13%, 14%, 15%, 16%, 17%, 18%, 19% or 20%, followed by a wash in 5×SSC, containing 0.1% (w/v) sodium dodecyl sulfate, at 55° C. to 65° C.

As used herein, “promoter” refers to a DNA sequence capable of controlling the expression of a coding sequence or functional RNA. The promoter sequence consists of proximal and more distal upstream elements, the latter elements often referred to as enhancers. Accordingly, an “enhancer” is a DNA sequence that can stimulate promoter activity, and may be an innate element of the promoter or a heterologous element inserted to enhance the level or tissue specificity of a promoter. Promoters may be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental conditions. It is further recognized that since in most cases the exact boundaries of regulatory sequences have not been completely defined, DNA fragments of some variation may have identical promoter activity.

As used herein, “terminator” generally refers to a section of DNA sequence that marks the end of a gene or operon in genomic DNA and is capable of stopping transcription. Terminators may be derived in their entirety from a native gene, or be composed of different elements derived from different terminators found in nature, or even comprise synthetic DNA segments. It is understood by those skilled in the art that different terminators may direct the expression of a gene in different tissues or cell types, or at different stages of development, or in response to different environmental conditions.

As used herein, the phrases “recombinant construct”, “expression construct”, “expression cassette”, “chimeric construct”, “construct”, and “recombinant DNA construct” are used interchangeably herein. A recombinant construct comprises an artificial combination of nucleic acid fragments, e.g., regulatory and coding sequences that are not found together in nature. For example, a chimeric construct may comprise regulatory sequences and coding sequences that are derived from different sources, or regulatory sequences and coding sequences derived from the same source, but arranged in a manner different than that found in nature. Such construct may be used by itself or may be used in conjunction with a vector. If a vector is used then the choice of vector is dependent upon the method that will be used to transform host cells as is well known to those skilled in the art. For example, a plasmid vector can be used. The skilled artisan is well aware of the genetic elements that must be present on the vector in order to successfully transform, select and propagate host cells comprising any of the isolated nucleic acid fragments of the disclosure. The skilled artisan will also recognize that different independent transformation events will result in different levels and patterns of expression (Jones et al., (1985) EMBO J. 4:2411-2418; De Almeida et al., (1989) Mol. Gen. Genetics 218:78-86), and thus that multiple events must be screened in order to obtain lines displaying the desired expression level and pattern. Such screening may be accomplished by Southern analysis of DNA, Northern analysis of mRNA expression, immunoblotting analysis of protein expression, or phenotypic analysis, among others. Vectors can be plasmids, viruses, bacteriophages, pro-viruses, phagemids, transposons, artificial chromosomes, and the like, that replicate autonomously or can integrate into a chromosome of a host cell. A vector can also be a naked RNA polynucleotide, a naked DNA polynucleotide, a polynucleotide composed of both DNA and RNA within the same strand, a poly-lysine-conjugated DNA or RNA, a peptide-conjugated DNA or RNA, a liposome-conjugated DNA, or the like, that is not autonomously replicating. As used herein, the term “expression” refers to the production of a functional end-product e.g., an mRNA or a protein (precursor or mature).

“Operably linked” means in this context the sequential arrangement of the promoter polynucleotide according to the disclosure with a further oligo- or polynucleotide, resulting in transcription of said further polynucleotide.

The term “product of interest” or “biomolecule” as used herein refers to any product produced by microbes from feedstock. In some cases, the product of interest may be a small molecule, enzyme, peptide, amino acid, organic acid, synthetic compound, fuel, alcohol, pharmaceutical, etc. For example, the product of interest or biomolecule may be any primary or secondary extracellular metabolite. The primary metabolite may be, inter alia, ethanol, citric acid, lactic acid, glutamic acid, glutamate, lysine, threonine, tryptophan and other amino acids, vitamins, polysaccharides, etc. The secondary metabolite may be, inter alia, an antibiotic compound like penicillin, or an immunosuppressant like cyclosporin A, a plant hormone like gibberellin, a statin drug like lovastatin, a fungicide like griseofulvin, etc. The product of interest or biomolecule may also be any intracellular component produced by a microbe, such as: a microbial enzyme, including: catalase, amylase, protease, pectinase, glucose isomerase, cellulase, hemicellulase, lipase, lactase, streptokinase, and many others. The intracellular component may also include recombinant proteins, such as: insulin, hepatitis B vaccine, interferon, granulocyte colony-stimulating factor, streptokinase and others. The product of interest may also refer to a “protein of interest”.

The term “protein of interest” generally refers to any polypeptide that is desired to be expressed in a filamentous fungus. Such a protein can be an enzyme, a substrate-binding protein, a surface-active protein, a structural protein, or the like, and can be expressed at high levels, and can be for the purpose of commercialization. The protein of interest can be encoded by an endogenous gene or a heterologous gene relative to the variant strain and/or the parental strain. The protein of interest can be expressed intracellularly or as a secreted protein. If the protein of interest is not naturally secreted, the polynucleotide encoding the protein may be modified to have a signal sequence in accordance with techniques known in the art. The proteins, which are secreted may be endogenous proteins which are expressed naturally, but can also be heterologous.

Heterologous means that the gene encoded by the protein is not produced under native condition in the filamentous fungal host cell. Examples of enzymes which may be produced by the filamentous fungi of the disclosure are carbohydrases, e.g. cellulases such as endoglucanases, beta-glucanases, cellobiohydrolases or beta-glucosidases, hemicellulases or pectinolytic enzymes such as xylanases, xylosidases, mannanases, galactanases, galactosidases, rhamnogalacturonases, arabanases, galacturonases, lyases, or amylolytic enzymes; phosphatases such as phytases, esterases such as lipases, proteolytic enzymes, oxidoreductases such as oxidases, transferases, or isomerases.

The term “carbon source” generally refers to a substance suitable to be used as a source of carbon for cell growth. Carbon sources include, but are not limited to, biomass hydrolysates, starch, sucrose, cellulose, hemicellulose, xylose, and lignin, as well as monomeric components of these substrates. Carbon sources can comprise various organic compounds in various forms, including, but not limited to polymers, carbohydrates, acids, alcohols, aldehydes, ketones, amino acids, peptides, etc. These include, for example, various monosaccharides such as glucose, dextrose (D-glucose), maltose, oligosaccharides, polysaccharides, saturated or unsaturated fatty acids, succinate, lactate, acetate, ethanol, etc., or mixtures thereof. Photosynthetic organisms can additionally produce a carbon source as a product of photosynthesis. In some embodiments, carbon sources may be selected from biomass hydrolysates and glucose.

The term “feedstock” is defined as a raw material or mixture of raw materials supplied to a microorganism or fermentation process from which other products can be made. For example, a carbon source, such as biomass or the carbon compounds derived from biomass are a feedstock for a microorganism that produces a product of interest (e.g. small molecule, peptide, synthetic compound, fuel, alcohol, etc.) in a fermentation process. However, a feedstock may contain nutrients other than a carbon source.

The term “volumetric productivity” or “production rate” is defined as the amount of product formed per volume of medium per unit of time. Volumetric productivity can be reported in gram per liter per hour (g/L/h).

The term “specific productivity” is defined as the rate of formation of the product. Specific productivity is herein further defined as the specific productivity in gram product per gram of cell dry weight (CDW) per hour (g/g CDW/h). Using the relation of CDW to OD₆₀₀ for the given microorganism specific productivity can also be expressed as gram product per liter culture medium per optical density of the culture broth at 600 nm (OD) per hour (g/L/h/OD).

The term “yield” is defined as the amount of product obtained per unit weight of raw material and may be expressed as g product per g substrate (g/g). Yield may be expressed as a percentage of the theoretical yield. “Theoretical yield” is defined as the maximum amount of product that can be generated per a given amount of substrate as dictated by the stoichiometry of the metabolic pathway used to make the product.

The term “titre” or “titer” is defined as the strength of a solution or the concentration of a substance in solution. For example, the titre of a product of interest (e.g. small molecule, peptide, synthetic compound, fuel, alcohol, etc.) in a fermentation broth is described as g of product of interest in solution per liter of fermentation broth (g/L).

The term “total titer” is defined as the sum of all product of interest produced in a process, including but not limited to the product of interest in solution, the product of interest in gas phase if applicable, and any product of interest removed from the process and recovered relative to the initial volume in the process or the operating volume in the process.

As used herein, the term “library” refers to collections of genetic perturbations according to the present disclosure. In some embodiments, the libraries of the present disclosure may manifest as i) a collection of genetic constructs encoding for the aforementioned series of genetic elements, or ii) host cell strains comprising said genetic elements. In some embodiments, the libraries of the present disclosure may refer to collections of individual elements (e.g., collections of terminators for SNPs for SNPswap libraries).

As used herein, the term “SNP” refers to Small Nuclear Polymorphism(s). In some embodiments, SNPs of the present disclosure should be construed broadly, and include single nucleotide polymorphisms, sequence insertions, deletions, inversions, and other sequence replacements. As used herein, the term “non-synonymous” or non-synonymous SNPs” refers to mutations that lead to coding changes in host cell proteins.

Overview

The present disclosure circumvent limitations described above by providing a high-throughput method for transforming filamentous fungal cells or protoplasts derived therefrom, purifying homokaryotic transformants and screening purified transformants. In general, the methods and systems described herein entail preparation of protoplasts from filamentous fungal cells, transformation of the prepared protoplasts, purification of protoplasts containing a single nucleus by altering the growth conditions used to prepare mycelia for protoplast preparation. Strain purification is achieved through selection and counter-selection, and, optionally, screening purified transformants possessing the correct phenotype and/or producing products of interest. The products of interest can be produced at a desired yield, productivity or titer. Preferably, protoplasts are used, but the method is applicable to other fungal cell types. In some cases, the methods and systems provided herein are high-throughput. In some cases, the methods and systems provided herein comprise steps that are semi-automated (e.g., transformation or selection, counterselection). In some cases, the methods and systems provided herein comprise steps that fully automated. In some cases, the methods and systems provided herein are high-throughput and the steps therein are semi-automated (e.g., transformation or selection, counterselection) or fully automated. As used herein, high-throughput can refers to any partially- or fully-automated method provided herein that is capable of evaluating about 1,000 or more transformants per day, and particularly to those methods capable of evaluating 5,000 or more transformants per day, and most particularly to methods capable of evaluating 10,000 or more transformants per day. Moreover, suitable volumes in which the method is performed are those of commercially available (deep well) microtiter plates, i.e. smaller than 1 ml, preferably smaller than 500 ul, more preferably smaller than 250 ul, most preferably from 1.5 ul to 250 ul, still most preferably from 10 ul to 100 ul.

The filamentous fungal cells used to prepare the protoplasts can be any filamentous fungus strains known in the art or described herein including holomorphs, teleomorphs or anamorphs thereof. The preparation of the protoplasts can be performed using those described herein or any known method in the art for preparing protoplasts.

Transformation of the protoplasts can be with at least one polynucleotide designed to integrate into a pre-determined locus in the filamentous fungal genome as provided herein. In a preferred embodiment, the protoplasts are co-transformed with at least two polynucleotides as provided herein such that each polynucleotide construct is designed to integrate into a different pre-determined locus in the filamentous fungal genome. In another preferred embodiment, a split marker transformation system is utilized. A pre-determined locus can be for a target filamentous fungal gene (e.g., a gene whose protein product is involved in citric acid production) or a selectable marker gene present in the filamentous fungal genome. A polynucleotide for use in transforming (e.g. via split marker design systems) or co-transforming protoplasts using the methods or systems provided herein can comprise sequence of a target filamentous fungal gene (e.g., a gene whose protein product is involved in citric acid production) comprising or containing a mutation and/or a genetic control element(s). The mutation can be a small nuclear polymorphism(s) such as a single nucleotide polymorphism, sequence insertions, deletions, inversions, and other sequence replacements. The genetic control element can be a promoter sequence (endogenous or heterologous) and/or a terminator sequence (endogenous or heterologous). A polynucleotide for use in transforming (e.g. via split marker design systems) or co-transforming protoplasts using the methods or systems provided herein can comprise sequence of a selectable marker gene. In one embodiment, the methods and systems provided herein entail co-transformation of protoplasts provided herein with two polynucleotides such that a first polynucleotide comprise sequence of a target filamentous fungal gene (e.g., a gene whose protein product is involved in citric acid production) comprising or containing a mutation and/or a genetic control element(s), while a second polynucleotide comprises sequence of a selectable marker gene. Further to this embodiment, the second polynucleotide can be designed to integrate into an additional selectable marker gene in the protoplast genome, while the first polynucleotide can be designed to integrate into the locus for the target filamentous fungal gene or, alternatively, into the locus of yet a further selectable marker gene. A selectable marker gene in any of the embodiments provided herein can be any of the selectable marker genes described herein. In other embodiments, a split marker design is utilized instead of the co-transformation method.

The disclosure also provides a method for preparing and storing a plurality of protoplasts from filamentous fungal cells. The method can entail removing cell walls from the filamentous fungal cells in the fungal culture, isolating the protoplasts, and resuspending the isolated protoplasts in a mixture comprising at least dimethyl sulfoxide (DMSO) and storing the isolated protoplasts. Storage can be for at least 1, 2, 3, 4, 5, 6, 7, 8, 9, 10, 11, 12 or 24 hours. Storage can be for at least 1, 7, 14, 30 or more days. Storage can be for at least 3, 6, 12, or more months. Storage can be at 4, −20 or −80° C. The fungal culture can be a culture with a volume of at least 500 ml, 1 liter, 2 liters, 3 liters, 4 liters or 5 liters. The filamentous fungal cells can be any filamentous fungus provided herein or known in the art. Prior to preparation of the protoplasts the fungal culture can be grown for at least 6, 8, 10, 12, 14, 16, 18, 20, 22 or 24 hours. In one embodiment, the fungal culture is grown under conditions whereby at least 70% of the protoplasts are homokaryotic following preparation of the protoplasts. In another embodiment, removing the cell walls is performed by enzymatic digestion. The enzymatic digestion can be performed with mixture of enzymes comprising a beta-glucanase and a polygalacturonase. The enzymatic digestion can be performed with VinoTaste concentrate. In yet another embodiment, the method further comprises adding polyethylene glycol (PEG) to the mixture comprising DMSO prior to storing the protoplasts. The PEG can be added to a final concentration of 50%, 40%, 30%, 20%, 15%, 10%, 5% or less. In still another embodiment, the method further comprises distributing the protoplasts into microtiter plates prior to storing the protoplasts. The microtiter plate can be a 6 well, 12 well, 24 well, 96 well, 384 well or 1536 well plate.

Filamentous Fungal Host Cells

In one embodiment, the methods and systems provided herein use fungal elements derived from filamentous fungus that are capable of being readily separated from other such elements in a culture medium, and are capable of reproducing itself. For example, the fungal elements can be a spore, propagule, hyphal fragment, protoplast or micropellet. In a preferred embodiment, the systems and methods provided herein utilize protoplasts derived from filamentous fungus. Suitable filamentous fungi host cells include, for example, any filamentous forms of the division Ascomycota, Deuteromycota, Zygomycota or Fungi imperfecti. Suitable filamentous fungi host cells include, for example, any filamentous forms of the subdivision Eumycotina. (see, e.g., Hawksworth et al., In Ainsworth and Bisby's Dictionary of The Fungi, 8th edition, 1995, CAB International, University Press, Cambridge, UK, which is incorporated herein by reference). In certain illustrative, but non-limiting embodiments, the filamentous fungal host cell may be a cell of a species of: Achlya, Acremonium, Aspergillus, Aureobasidium, Bjerkandera, Ceriporiopsis, Cephalosporium, Chrysosporium, Cochliobolus, Corynascus, Cryphonectria, Cryptococcus, Coprinus, Coriolus, Diplodia, Endothis, Filibasidium, Fusarium, Gibberella, Gliocladium, Humicola, Hypocrea, Myceliophthora (e.g., Myceliophthora thermophila), Mucor, Neurospora, Penicillium, Podospora, Phlebia, Piromyces, Pyricularia, Rhizomucor, Rhizopus, Schizophyllum, Scytalidium, Sporotrichum, Talaromyces, Thermoascus, Thielavia, Tramates, Tolypocladium, Trichoderma, Verticillium, Volvariella, or teleomorphs, or anamorphs, and synonyms or taxonomic equivalents thereof. In one embodiment, the filamentous fungus is selected from the group consisting of A. nidulans, A. oryzae, A. sojae, and Aspergilli of the A. niger Group. In a preferred embodiment, the filamentous fungus is Aspergillus niger.

In another embodiment the disclosure provides specific mutants of the fungal species are used for the methods and systems provided herein. In one embodiment, specific mutants of the fungal species are used which are suitable for the high-throughput and/or automated methods and systems provided herein. Examples of such mutants can be strains that protoplast very well; strains that produce mainly or, more preferably, only protoplasts with a single nucleus; strains that regenerate efficiently in microtiter plates, strains that regenerate faster and/or strains that take up polynucleotide (e.g., DNA) molecules efficiently, strains that produce cultures of low viscosity such as, for example, cells that produce hyphae in culture that are not so entangled as to prevent isolation of single clones and/or raise the viscosity of the culture, strains that have reduced random integration (e.g., disabled non-homologous end joining pathway) or combinations thereof. In yet another embodiment, a specific mutant strain for use in the methods and systems provided herein can be strains lacking a selectable marker gene such as, for example, uridine-requiring mutant strains. These mutant strains can be either deficient in orotidine 5 phosphate decarboxylase (OMPD) or orotate p-ribosyl transferase (OPRT) encoded by the pyrG or pyrE gene, respectively (T. Goosen et al., Curr Genet. 1987, 11:499 503; J. Begueret et al., Gene. 1984 32:487 92.

In one embodiment, specific mutant strains for use in the methods and systems provided herein are strains that possess a compact cellular morphology characterized by shorter hyphae and a more yeast-like appearance. Examples of such mutants can be filamentous fungal cells with altered gas1 expression as described in US20140220689.

In still another embodiment, mutant strains for use in the methods and systems provided herein are modified in their DNA repair system in such a way that they are extremely efficient in homologous recombination and/or extremely inefficient in random integration. The efficiency of targeted integration of a nucleic acid construct into the genome of the host cell by homologous recombination, i.e. integration in a predetermined target locus, can be increased by augmented homologous recombination abilities and/or diminished non-homologous recombination abilities of the host cell. Augmentation of homologous recombination can be achieved by overexpressing one or more genes involved in homologous recombination (e.g., Rad51 and/or Rad52 protein). Removal, disruption or reduction in the activity of one or more non-homologous recombination pathways (e.g., the canonical non-homologous end joining (NHEJ) pathway, the Alternative NHEJ or microhomology-mediated end-joining (Alt-NHEJ/MMEJ) pathway and/or the polymerase theta mediated end-joining (TMEJ) pathway) in the host cells of the present disclosure can be achieved by any method known in that art such as, for example, by use of an antibody, a chemical inhibitor, a protein inhibitor, a physical inhibitor, a peptide inhibitor, or an anti-sense or RNAi molecule directed against a component of a specific non-homologous recombination (NHR) pathway (e.g., the NHEJ pathway, the Alt-NHEJ/MMEJ pathway and/or the TMEJ pathway). In one embodiment, the activity of a single non-homologous end joining pathway is inhibited or reduced. In another embodiment, the activity of a combination of non-homologous end-joining pathways are inhibited or reduced such that the activity of one of the non-homologous end-joining pathways remains intact. In yet another embodiment, the activity of every non-homologous end-joining pathways are reduced or inhibited.

Examples of components of the NHEJ pathway that can be targeted for inhibition or reduction of activity alone or in combination can include, but are not limited to yeast KU70 or yeast KU80 or homologues or orthologs thereof. Examples of components of the Alt-NHEJ/MMEJ pathway that can be targeted for inhibition or a reduction in activity alone or in combination can include, but are not limited to a Polq gene, a Mre 11 gene, a XPF-ERCC1 gene or homologues or orthologs thereof. An example of a component of the TMEJ pathway that can be targeted for inhibition or a reduction in activity can include, but is not limited to a Polq gene or homologues or orthologs thereof. In some cases, a host-cell for use in the methods provided herein can be deficient in one or more genes (e.g., yeast ku70, ku80 or homologues or orthologs thereof) of the NHEJ pathway. Examples of such mutants are cells with a deficient hdfA or hdfB gene as described in WO 05/95624. In some cases, a host-cell for use in the methods provided herein can be deficient in one or more genes of the Alternative NHEJ or microhomology-mediated end-joining (Alt-NHEJ/MMEJ) pathway and/or TMEJ pathway. Examples of such mutants are cells with that lack Polq gene or possess a mutant Polq gene as described in Wyatt et al. Essential roles for Polymerase θ mediated end-joining in repair of chromosome breaks Mol Cell. 2016 Aug. 18; 63(4): 662-673.

Examples of chemical inhibitors for use in inhibiting one or more NHR pathways (e.g., the NHEJ pathway, the Alt-NHEJ/MMEJ pathway and/or the TMEJ pathway) in host cells for use in the methods provided herein can be W7, chlorpromazine, vanillin, Nu7026, Nu7441, mirin, SCR7, AG14361 or a combination thereof. Further, inhibition of the NHEJ pathway can be achieved using chemical inhibitors such as described in Arras S M D, Fraser J A (2016), “Chemical Inhibitors of Non-Homologous End Joining Increase Targeted Construct Integration in Cryptococcus neoformans” PloS ONE 11 (9): e0163049, the contents of which are hereby incorporated by reference.

In a preferred embodiment, a mutant strain of filamentous fungal cell for use in the methods and systems provided herein have a disabled or reduced non-homologous end-joining pathway (either the NHEJ pathway, the Alt-NHEJ/MMEJ pathway or the TMEJ pathway or a combination thereof) and possess a yeast-like, non-mycelium forming phenotype when grown in culture (e.g., submerged culture).

Protoplasting Methods

In one embodiment, the methods and systems provided herein require the generation of protoplasts from filamentous fungal cells. Suitable procedures for preparation of protoplasts can be any known in the art including, for example, those described in EP 238,023 and Yelton et al. (1984, Proc. Natl. Acad. Sci. USA 81:1470-1474). In one embodiment, protoplasts are generated by treating a culture of filamentous fungal cells with one or more lytic enzymes or a mixture thereof. The lytic enzymes can be a beta-glucanase and/or a polygalacturonase. In one embodiment, the enzyme mixture for generating protoplasts is VinoTaste concentrate. Following enzymatic treatment, the protoplasts can be isolated using methods known in the art. For example, undigested hyphal fragments can be removed by filtering the mixture through a porous barrier (such as Miracloth) in which the pores range in size from 20-100 microns. The filtrate containing the protoplasts can then be centrifuged at moderate speeds to cause the protoplasts to pellet to the bottom of the centrifuge tube. Alternatively, a buffer of substantially lower osmotic strength can be gently applied to the surface of the filtered protoplasts. This layered preparation can then be centrifuged, which can cause the protoplasts to accumulate at a layer in the tube in which they are neutrally buoyant. Protoplasts can then be isolated from this layer for further processing. Following protoplast isolation, the remaining enzyme containing buffer can be removed by resuspending the protoplasts in an osmotic buffer (typically 1M sorbitol buffered using TRIS) and recollected by centrifugation. This step can be repeated. After sufficient removal of the enzyme containing buffer, the protoplasts can be resuspended in osmotically stabilized buffer also containing Calcium chloride. In one embodiment, the protoplasts are resuspended to a final concentration between 1-3*10⁷ protoplasts per ml.

The pre-cultivation and the actual protoplasting step can be varied to optimize the number of protoplasts and the transformation efficiency. A typical preparation can be to inoculate 100 ml of rich media such as YPD with 10⁶ spores/ml and incubate between 14-18 hours. Many of these parameters may be varied. For example, there can be variations of inoculum size, inoculum method, pre-cultivation media, pre-cultivation times, pre-cultivation temperatures, mixing conditions, washing buffer composition, dilution ratios, buffer composition during lytic enzyme treatment, the type and/or concentration of lytic enzyme used, the time of incubation with lytic enzyme, the protoplast washing procedures and/or buffers, the concentration of protoplasts and/or polynucleotide and/or transformation reagents during the actual transformation, the physical parameters during the transformation, the procedures following the transformation up to the obtained transformants.

Protoplasts can be resuspended in an osmotic stabilizing buffer. The composition of such buffers can vary depending on the species, application and needs. However, typically these buffers contain either an organic component like sucrose, citrate, mannitol or sorbitol between 0.5 and 2 M. More preferably between 0.75 and 1.5 M; most preferred is 1 M. Otherwise these buffers contain an inorganic osmotic stabilizing component like KCl, (NH₄)₂SO₄, MgSO₄, NaCl or MgCl₂ in concentrations between 0.1 and 1.5 M. Preferably between 0.2 and 0.8 M; more preferably between 0.3 and 0.6 M, most preferably 0.4 M. The most preferred stabilizing buffers are STC (sorbitol, 0.8 M; CaCl.sub.2, 25 mM; Tris, 25 mM; pH 8.0) or KCl-citrate (KCl, 0.3-0.6 M; citrate, 0.2% (w/v)). The protoplasts can be used in a concentration between 1×10⁵ and 1×10¹⁰ cells/ml. Preferably, the concentration is between 1×10⁶ and 1×10⁹; more preferably the concentration is between 1×10⁷ and 5×10⁸; most preferably the concentration is 1×10⁸ cells/ml. DNA is used in a concentration between 0.01 and 10 ug; preferably between 0.1 and 5 ug, even more preferably between 0.25 and 2 ug; most preferably between 0.5 and 1 ug. To increase the efficiency of transfection carrier DNA (as salmon sperm DNA or non-coding vector DNA) may be added to the transformation mixture.

In one embodiment, following generation and subsequent isolation, the protoplasts are mixed with one or more cryoprotectants. The cryoprotectants can be glycols, dimethyl sulfoxide (DMSO), polyols, sugars, 2-Methyl-2,4-pentanediol (MPD), polyvinylpyrrolidone (PVP), methylcellulose, C-linked antifreeze glycoproteins (C-AFGP) or combinations thereof. Glycols for use as cryoprotectants in the methods and systems provided herein can be selected from ethylene glycol, propylene glycol, polypropylene glycol (PEG), glycerol, or combinations thereof. Polyols for use as cryoprotectants in the methods and systems provided herein can be selected from propane-1,2-diol, propane-1,3-diol, 1,1,1-tris-(hydroxymethyl)ethane (THME), and 2-ethyl-2-(hydroxymethyl)-propane-1,3-diol (EHMP), or combinations thereof. Sugars for use as cryoprotectants in the methods and systems provided herein can be selected from trehalose, sucrose, glucose, raffinose, dextrose or combinations thereof. In one embodiment, the protoplasts are mixed with DMSO. DMSO can be mixed with the protoplasts at a final concentration of at least, at most, less than, greater than, equal to, or about 1%, 2%, 3%, 4%, 5%, 6%, 7%, 8%, 9%, 10%, 12.5%, 15%, 20%, 25%, 30%, 35%, 40%, 45%, 50%, 55%, 60%, 65%, 70%, or 75% w/v or v/v. The protoplasts/cryoprotectant (e.g., DMSO) mixture can be distributed to microtiter plates prior to storage. The protoplast/cryoprotectant (e.g., DMSO) mixture can be stored at any temperature provided herein for long-term storage (e.g., several hours, day(s), week(s), month(s), year(s)) as provided herein such as, for example −20° C. or −80° C. In one embodiment, an additional cryoprotectant (e.g., PEG) is added to the protoplasts/DMSO mixture. In yet another embodiment, the additional cryoprotectant (e.g., PEG) is added to the protoplast/DMSO mixture prior to storage. The PEG can be any PEG provided herein and can be added at any concentration (e.g., w/v or v/v) as provided herein. In one embodiment. the PEG solution is prepared as 40% w/v in STC buffer. 20% v/v of this 40% PEG-STC can then be added to the protoplasts. For example, 800 microliters of 1.25×10⁷ protoplasts would have 200 microliters of 40% PEG-STC giving a final volume of 1 ml. Seventy microliters of DMSO can then be added to this 1 ml to bring this prep to 7% v/v DMSO.

Transformation Methods

In one embodiment, the methods and systems provided herein require the transfer of nucleic acids to protoplasts derived from filamentous fungal cells as described herein. In another embodiment, the transformation utilized by the methods and systems provided herein is high-throughput in nature and/or is partially or fully automated as described herein. Further to this embodiment, the transformation is performed by adding constructs or expression constructs as described herein to the wells of a microtiter plate followed by aliquoting protoplasts generated by the methods provided herein to each well of the microtiter plate. Suitable procedures for transformation/transfection of protoplasts can be any known in the art including, for example, those described in international patent applications PCT/NL99/00618, PCT/EP99/202516, Finkelstein and Ball (eds.), Biotechnology of filamentous fungi, technology and products, Butterworth-Heinemann (1992), Bennett and Lasure (eds.) More Gene Manipulations in fungi, Academic Press (1991), Turner, in: Puhler (ed), Biotechnology, second completely revised edition, VHC (1992) protoplast fusion, and the Ca-PEG mediated protoplast transformation as described in EP635574B. Alternatively, transformation of the filamentous fungal host cells or protoplasts derived therefrom can also be performed by electroporation such as, for example, the electroporation described by Chakraborty and Kapoor, Nucleic Acids Res. 18:6737 (1990), Agrobacterium tumefaciens-mediated transformation, biolistic introduction of DNA such as, for example, as described in Christiansen et al., Curr. Genet. 29:100 102 (1995); Durand et al., Curr. Genet. 31:158 161 (1997); and Barcellos et al., Can. J. Microbiol. 44:1137 1141 (1998) or “magneto-biolistic” transfection of cells such as, for example, described in U.S. Pat. Nos. 5,516,670 and 5,753,477. In one embodiment, transformation of the filamentous fungal host cells or protoplasts derived therefrom is performed using a method utilizing shock-waves. The shock-wave method can be any shock-wave method known in art, such as, for example, the single pulse, underwater shock-wave method described by Denis Magaña-Ortiz, Nancy Coconi-Linares, Elizabeth Ortiz-Vazquez, Francisco Fernández, Achim M. Loske, Miguel A. Gómez-Lim (2013) A novel and highly efficient method for genetic transformation of fungi employing shock waves. Fungal Genetics and Biology. 56:9-16. The shock-wave method for use in the methods herein can also be the dual pulse shock waves as described by Loske A M, Fernández F, Magaña-Ortiz, Coconi-Linares N, Ortiz-Vazquez E, Gomez-Lim M A (2014) Tandem shock waves to enhance genetic transformation of Aspergillus niger. Ultrasonics. 54(6):1656-62. In one embodiment, the transformation procedure used in the methods and systems provided herein is one amendable to being high-throughput and/or automated as provided herein such as, for example, PEG mediated transformation.

Transformation of the protoplasts generated using the methods described herein can be facilitated through the use of any transformation reagent known in the art. Suitable transformation reagents can be selected from Polyethylene Glycol (PEG), FUGENE® HD (from Roche), Lipofectamine® or OLIGOFECTAMINE® (from Invitrogen), TRANSPASS®D1 (from New England Biolabs), LYPOVEC® or LIPOGEN® (from Invivogen). In one embodiment, PEG is the most preferred transformation/transfection reagent. PEG is available at different molecular weights and can be used at different concentrations. Preferably, PEG 4000 is used between 10% and 60%, more preferably between 20% and 50%, most preferably at 40%. In one embodiment, the PEG is added to the protoplasts prior to storage as described herein.

Constructs for Transformation

In one embodiment, the methods and systems provided herein entail the transformation or transfection of filamentous fungal cells or protoplasts derived therefrom with at least one nucleic acid. The transformation or transfection can be using of the methods and reagents described herein. The generation of the protoplasts can be performed using any of the methods provided herein. The protoplast generation and/or transformation can be high-throughput and/or automated as provided herein. The nucleic acid can be DNA, RNA or cDNA. The nucleic acid can be a polynucleotide. The nucleic acid or polynucleotide for use in transforming a filamentous fungal cell or protoplast derived therefrom using the methods and systems provided herein can be an endogenous gene or a heterologous gene relative to the variant strain and/or the parental strain. The endogenous gene or heterologous gene can encode a product or protein of interest as described herein. As described herein, the protein of interest can refer to a polypeptide that is desired to be expressed in a filamentous fungus. Such a protein can be an enzyme, a substrate-binding protein, a surface-active protein, a structural protein, or the like, and can be expressed at high levels, and can be for the purpose of commercialization. The protein of interest can be expressed intracellularly or as a secreted protein. The endogenous gene or heterologous gene can comprise a mutation and/or be under the control of or operably linked to one or more genetic control or regulatory elements. The mutation can be any mutation provided herein such as, for example, an insertion, deletion, substitution and/or single nucleotide polymorphism. The one or more genetic control or regulatory elements can be a promoter sequence and/or a terminator sequence.

The promoter sequence and/or terminator sequence can be endogenous or heterologous relative to the variant strain and/or the parental strain. Promoter sequences can be operably linked to the 5′ termini of the sequences to be expressed. A variety of known fungal promoters are likely to be functional in the disclosed host strains such as, for example, the promoter sequences of C1 endoglucanases, the 55 kDa cellobiohydrolase (CBH1), glyceraldehyde-3-phosphate dehydrogenase A, C. lucknowense GARG 27K and the 30 kDa xylanase (Xy1F) promoters from Chrysosporium, as well as the Aspergillus promoters described in, e.g. U.S. Pat. Nos. 4,935,349; 5,198,345; 5,252,726; 5,705,358; and 5,965,384; and PCT application WO 93/07277. Terminator sequences can be operably linked to the 3′ termini of the sequences to be expressed. A variety of known fungal terminators are likely to be functional in the disclosed host strains. Examples are the A. nidulans trpC terminator, A. niger alpha-glucosidase terminator, A. niger glucoamylase terminator, Mucor miehei carboxyl protease terminator (see U.S. Pat. No. 5,578,463), Chrysosporium terminator sequences, e.g. the EG6 terminator, and the Trichoderma reesei cellobiohydrolase terminator.

In one embodiment, a protoplast generated from a filamentous fungal cell is co-transformed with two or more nucleic acids or polynucleotides. Further to this embodiment, at least one of the two or more polynucleotides is an endogenous gene or a heterologous gene relative to the filamentous fungal strain from which the protoplast was generated and at least one of the two or more polynucleotides is a gene for a selectable marker. The selectable marker gene can be any selectable marker as provided herein. In other embodiments, a split marker system is utilized.

In one embodiment, each nucleic acid or polynucleotide for use in transforming or transfecting a filamentous fungal cell or protoplast derived therefrom comprises sequence homologous to DNA sequence present in a pre-determined target locus of the genome of the filamentous fungal cell or protoplast derived therefrom that is to be transformed on either a 5′, a 3′ or both a 5′ and a 3′ end of the nucleic acid or polynucleotide. The nucleic acid or polynucleotide can be an endogenous gene or heterologous gene relative to the filamentous fungal cell used for transformation or a selectable marker gene such that sequence homologous to a pre-determined locus in the filamentous fungal host cell genome flanks the endogenous, heterologous, or selectable marker gene. In one embodiment, each nucleic acid or polynucleotide is cloned into a cloning vector using any method known in the art such as, for example, pBLUESCRIPT® (Stratagene). Suitable cloning vectors can be the ones that are able to integrate at the pre-determined target locus in the chromosomes of the filamentous fungal host cell used. Preferred integrative cloning vectors can comprise a DNA fragment, which is homologous to the DNA sequence to be deleted or replaced for targeting the integration of the cloning vector to this pre-determined locus. In order to promote targeted integration, the cloning vector can be linearized prior to transformation of the host cell or protoplasts derived therefrom. Preferably, linearization is performed such that at least one but preferably either end of the cloning vector is flanked by sequences homologous to the DNA sequence to be deleted or replaced. In some cases, short homologous stretches of DNA may be added for example via PCR on both sides of the nucleic acid or polynucleotide to be integrated. The length of the homologous sequences flanking the nucleic acid or polynucleotide sequence to be integrated is preferably less than 2 kb, even preferably less, than 1 kb, even more preferably less than 0.5 kb, even more preferably less than 0.2 kb, even more preferably less than 0.1 kb, even more preferably less than 50 bp and most preferably less than 30 bp. The length of the homologous sequences flanking the nucleic acid or polynucleotide sequence to be integrated can vary from about 30 bp to about 1000 bp, from about 30 bp to about 700 bp, from about 30 bp to about 500 bp, from about 30 bp to about 300 bp, from about 30 bp to about 200 bp, and from about 30 bp to about 100 bp. The nucleic acids or polynucleotides for use in transforming filamentous fungal cells or protoplasts derived therefrom can be present as expression cassettes. In one embodiment, the cloning vector is pUC19. Further to this embodiment, a cloning vector containing a marker sequence as provided herein can be associated with targeting sequence by building the construct through using a Gibson assembly as known in the art. Alternatively, the targeting sequence can be added by fusion PCR. Targeting sequence for co-transformation that is not linked to a marker may be amplified from genomic DNA.

In theory, all loci in the filamentous fungi genome could be chosen for targeted integration of the expression cassettes comprising nucleic acids or polynucleotides provided herein. Preferably, the locus wherein targeting will take place is such that when the wild type gene present at this locus has been replaced by the gene comprised in the expression cassette, the obtained mutant will display a change detectable by a given assay such as, for example a selection/counterselection scheme as described herein. In one embodiment, the protoplasts generated from filamentous fungal cells as described herein are co-transformed with a first construct or expression cassette and a second construct or expression cassette such that the first construct or expression cassette is designed to integrate into a first locus of the protoplast genome, while the second construct or expression cassette is designed to integrate into a second locus of the protoplast genome. To facilitate integration into the first locus and second locus, the first construct or expression cassette is flanked by sequence homologous to the first locus, while the second construct or expression cassette is flanked by sequence homologous to the second locus. In one embodiment, the first construct or expression cassette comprises sequence for an endogenous gene, while the second construct comprises sequence for a selectable marker gene. Further to this embodiment, the second locus contains sequence for an additional selectable marker gene present in the protoplast genome used in the methods and systems provided herein, while the first locus contains sequence for the endogenous target gene present in the protoplast genome used in the methods and systems provided herein. In a separate embodiment, the first construct or expression cassette comprises sequence for an endogenous gene or a heterologous gene, while the second construct comprises sequence for a first selectable marker gene. Further to this separate embodiment, the second locus contains sequence for a second selectable marker gene that is present in the protoplast genome used in the methods and systems provided herein, while the first locus contains sequence for a third selectable marker gene that is present in the protoplast genome used in the methods and systems provided herein. In each of the above embodiments, the endogenous gene and/or heterologous gene can comprise a mutation (e.g., SNP) and/or a genetic control or regulatory element as provided herein. In another aspect, a split marker system is utilized.

Split Marker Transformation

The “split marker” transformation, as described in Catlett, N. L., B. Lee, O. C. Yoder, and B. G. Turgeon (2003) “Split-Marker Recombination for Efficient Targeted Deletion of Fungal Genes,” Fungal Genetics Reports: Vol. 50, Article 4, utilizes fragments of a gene that confers a selectable phenotype. The individual fragments do not complement the mutation in the target strain or do not confer antibiotic resistance when they are integrated into a strain individually. Proper expression of the selectable marker occurs when the two fragments recombine and repair the resistance gene or the auxotrophic marker.

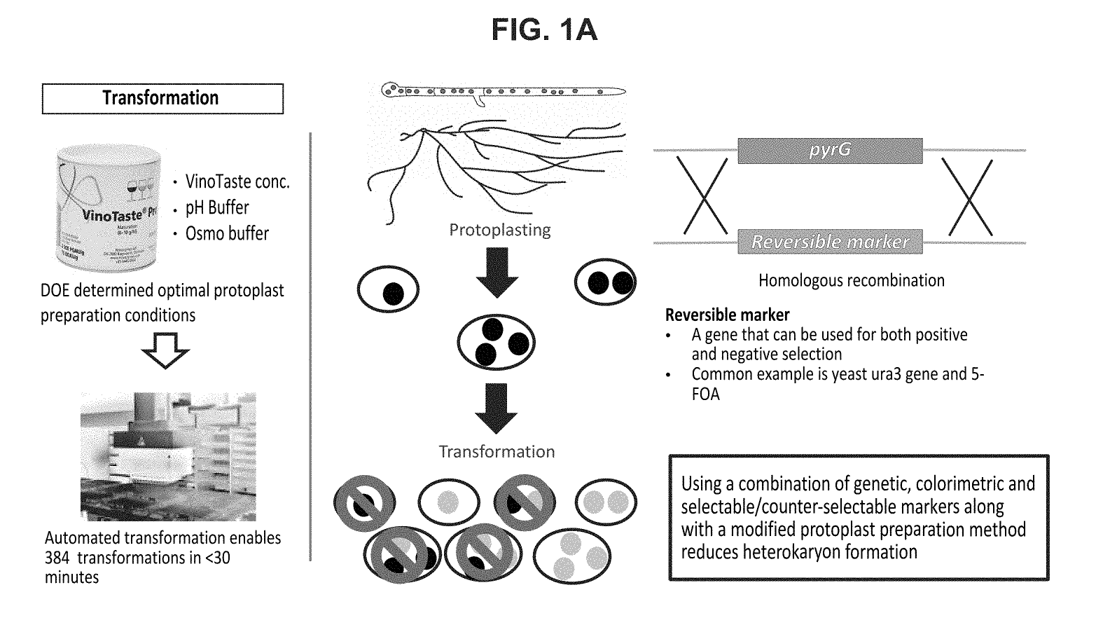

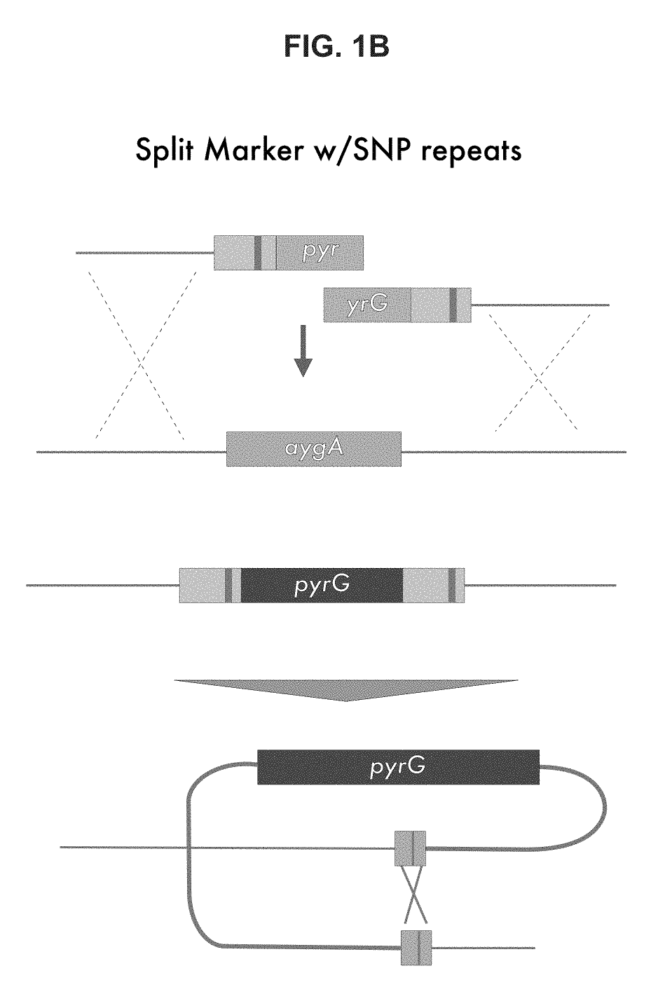

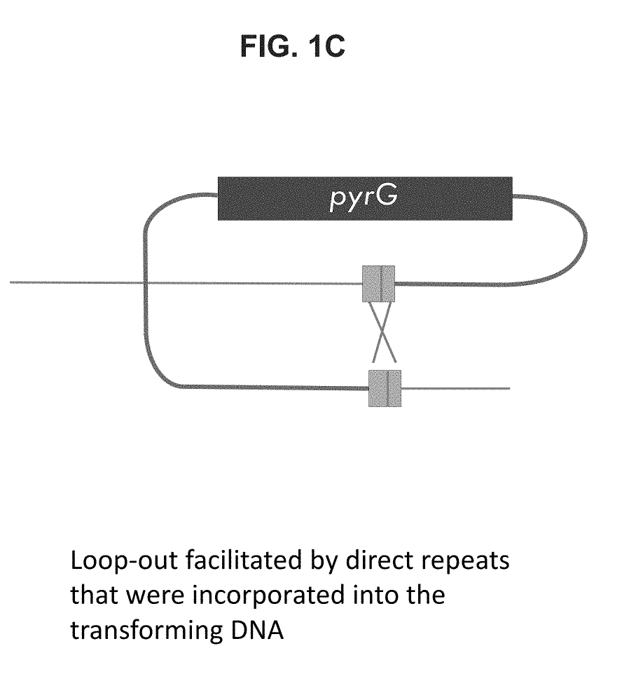

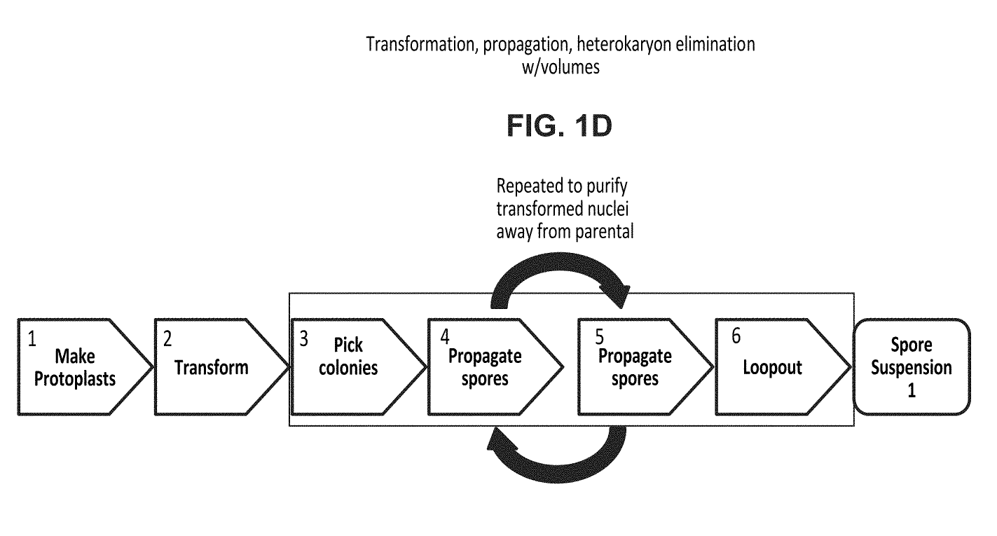

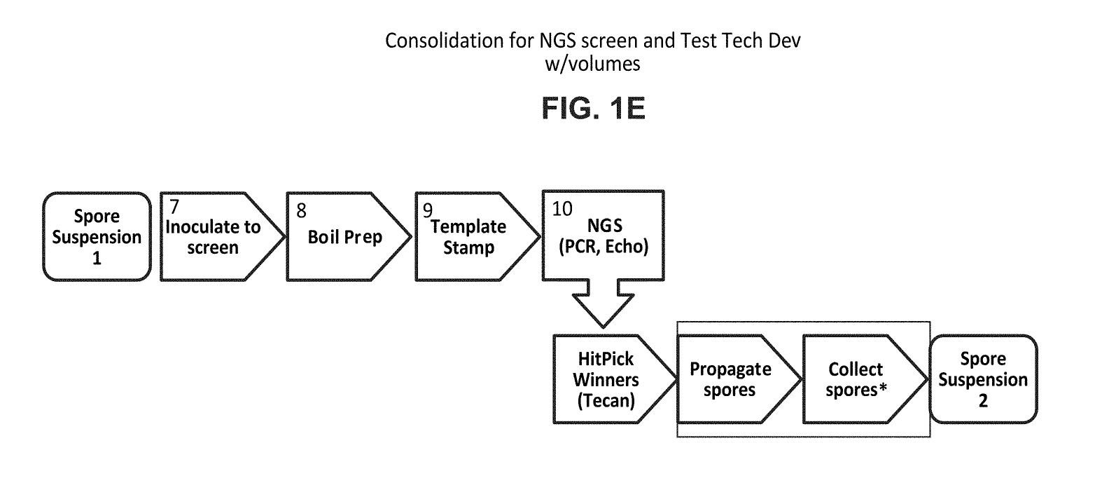

An example of split marker is diagrammed in FIG. 1B. In this example the marker gene is pyrG. The fragments of DNA represented are the 5′ half of the pyrG gene as “pyr” and the 3′ half as “yrG.” These constructs are used to complement a pyrG mutation in the target strain. If the pyr portion integrates without recombining with the yrG portion, there is no complementation. Direct repeats of a region of sequence which contains the desired SNP are add

Gene targeting using the split marker constructs occurs when DNA that is identical to the target locus are fused to each of the two components. For example, targeting a single base pair change is performed by fusing DNA corresponding to the 5′ region upstream of the SNP to the 5′ half of the split marker. The corresponding 3′ DNA is fused to the 3′ split marker. When these recombine in the cell through homologous integration the two direct repeats are at the SNP locus in the target strain that matches that in the SNP library. Completion of the SNP exchange occurs when the marker is looped out by homologous recombination between the two direct repeats.

Purification of Homokaryotic Protoplasts

As will be appreciated by those skilled in the art, protoplasts derived from filamentous fungal can often contain more than one nucleus such that subsequent transformation with an expression construct as provided herein can produce protoplasts that are heterokaryotic such that the expression construct is incorporated into only a subset of the multiple nuclei present in the protoplast. Strategies can be employed to increase the percentage of mononuclear protoplasts in a population of protoplasts from filamentous fungal host cells prior to transformation such as, for example, as described in Roncero et al., 1984, Mutat. Res. 125:195

A selectable marker can often encodes a gene product providing a specific type of resistance foreign to the non-transformed strain. This can be resistance to heavy metals, antibiotics or biocides in general. Prototrophy can also a useful selectable marker of the non-antibiotic variety. Auxotrophic markers can generate nutritional deficiencies in the host cells, and genes correcting those deficiencies can be used for selection.

There is a wide range of selection markers in use in the art and any or all of these can be applied to the methods and systems provided herein. The selectable marker genes for use herein can be auxotrophic markers, prototrophic markers, dominant markers, recessive markers, antibiotic resistance markers, catabolic markers, enzymatic markers, fluorescent markers, luminescent markers or combinations thereof. Examples of selectable/counterselectable markers for use in the methods provided herein include, but are not limited to: amdS (selection on acetamide/counterselection on fluoroacetamide), tk (thymidine kinase from Herpes virus; counterselection on 5-fluoro-2′-deoxyuridine), ble (belomycin-phleomycin resistance), hyg (hygromycinR), nat (nourseotricin R), pyrG (selection on uracil+uridine/counterselection on 5′ FOA), niaD or niaA (selection on nitrate/counterselection on chlorate), sutB (selection on sulphate/counterselection on selenate), eGFP (Green Fluorescent Protein) and all the different color variants, aygA (colorimetric marker), met3 (selection on methionine/counterselection on selenate), sC/metA (selection on sulfate/counterselection on selenate), pyrE (orotate P-ribosyl transferase), trpC (anthranilate synthase), argB (ornithine carbamoyltransferase), bar (phosphinothricin acetyltransferase), mutant acetolactate synthase (sulfonylurea resistance), and neomycin phosphotransferase (aminoglycoside resistance).

Another embodiment of the disclosure entails the use of two or more selection markers active in filamentous fungi. There is a wide range of combinations of selection markers that can be used and all of these can be applied in the selection/counterselection scheme provided herein. For example, the selection/counterselection scheme can utilize a combination of auxotrophic markers, prototrophic markers, dominant markers, recessive markers, antibiotic resistance markers, catabolic markers, enzymatic markers, fluorescent markers, and luminescent markers. A first marker can be used to select in the forward mode (i.e., if active integration has occurred), while additional markers can be used to select in the reverse mode (i.e., if active integration at the right locus has occurred). Selection/counterselection can be carried out by cotransformation such that a selection marker can be on a separate vector or can be in the same nucleic acid fragment or vector as the endogenous or heterologous gene as described herein.

In one embodiment, the homokaryotic protoplast purification scheme of the disclosure entails co-transforming protoplasts generated from filamentous fungal host cells with a first construct comprising sequence for an endogenous gene or heterologous gene and a second construct comprising sequence for a first selectable marker gene such that the first construct is directed to a first locus of the protoplast genome that comprises sequence for a target gene to be removed or inactivated, while the second construct is directed to a second locus of the protoplast genome that comprises sequence for a second selectable marker gene. In one embodiment, the first construct comprises sequence for an endogenous gene or heterologous gene and the target gene to be removed or inactivated is for a third selectable marker gene. In a separate embodiment, the first construct comprises a sequence for an endogenous gene and the target gene to be removed or inactivated is the copy of the endogenous gene present in the genome of the protoplast prior to transformation. As described herein, the endogenous gene or heterologous gene of the first construct can comprise a mutation (e.g., SNP) and/or a genetic regulatory or control element (e.g., promoter and/or terminator). The first, second and/or third selectable marker can be any auxotrophic markers, prototrophic markers, dominant markers, recessive markers, antibiotic resistance markers, catabolic markers, enzymatic markers, fluorescent markers, luminescent markers known in the art and/or described herein. To be directed to a specific locus each of the constructs is flanked by nucleotides homologous to the desired locus in the protoplast genome as described herein.

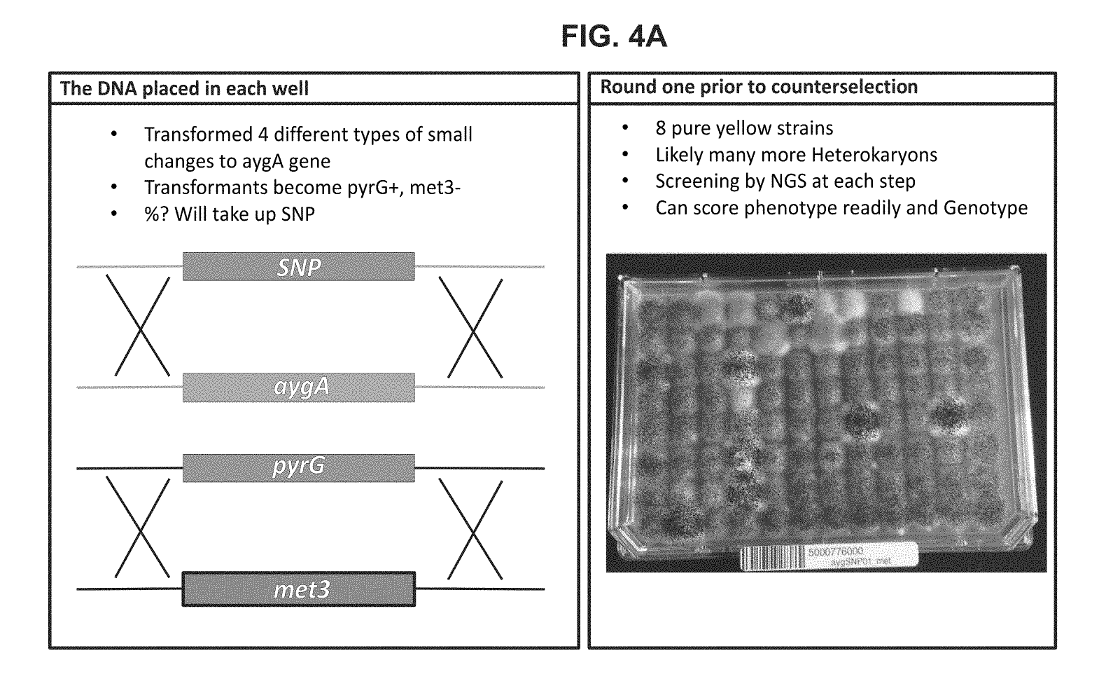

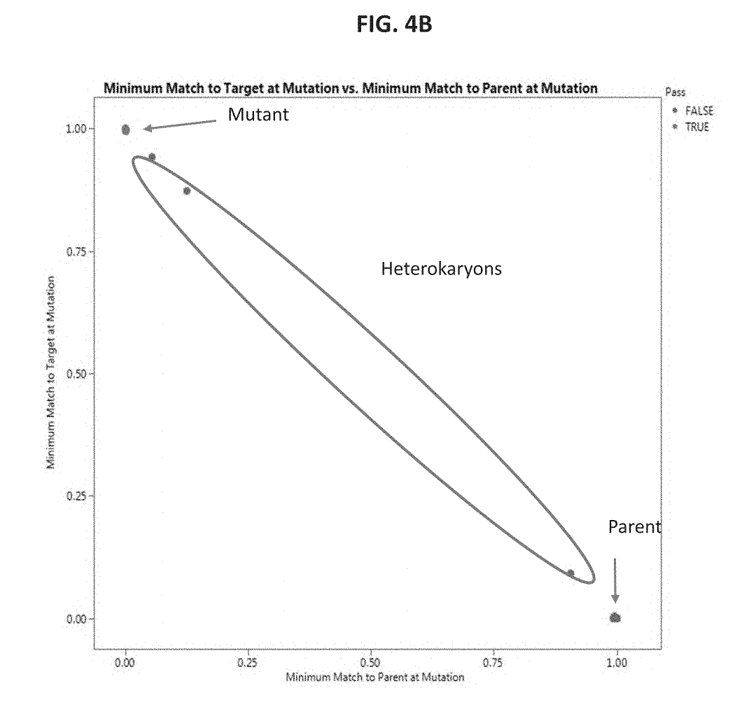

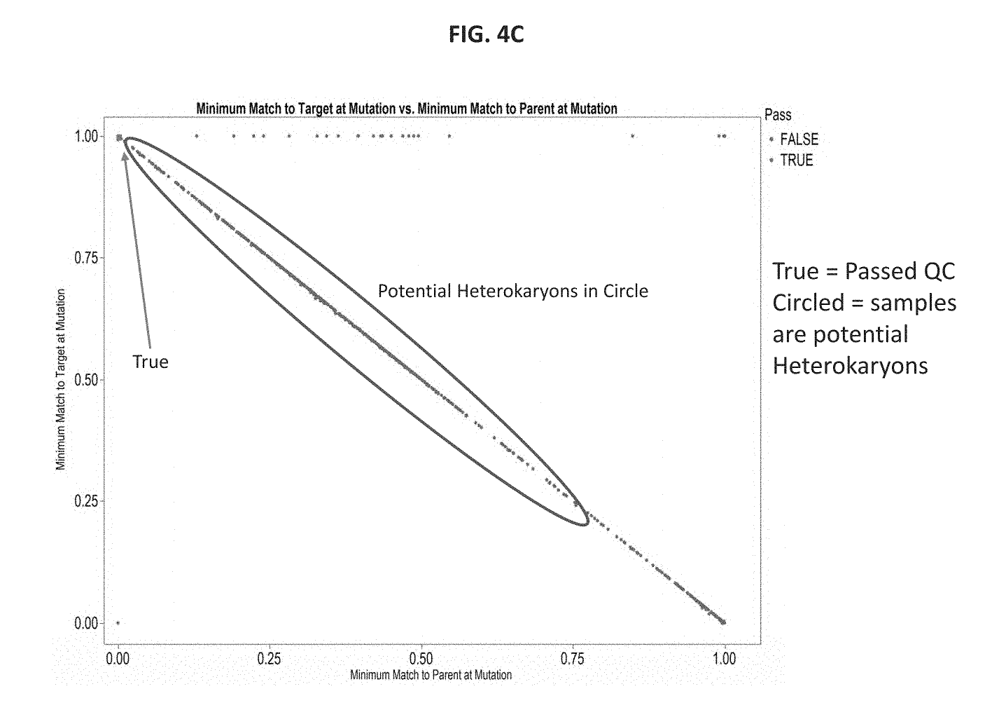

An example of the embodiment where the first construct comprises sequence for an endogenous gene or heterologous gene and the target gene to be removed or inactivated is for a third selectable marker gene is shown in FIG. 4A. In this example, the first construct comprises sequence for an endogenous gene involved in citric acid production in filamentous fungus that comprises a SNP that is integrated into the locus for the colorimetric selectable marker gene aygA, while the second construct comprises sequence for the auxotrophic marker gene pyrG that is integrated into the locus for the auxotrophic marker gene met3. In this example, the filamentous fungal host cell is pyrG negative or uracil auxotrophic. Accordingly, purification of homokaryotic protoplast transformants entails growing said transformants on minimal media lacking uracil. As shown in FIG. 4A, homokaryotic transformants will not only be uracil prototrophs, but will also be pure yellow in color, indicting incorporation of the pyrG gene and removal of the aygA gene. Counterselection and removal of any residual heterokaryotic colonies can be accomplished by subsequently plating the transformants on minimal media (with or without uracil) that contains selenate, whereby transformants with met3+nucleic will die in the presence of selenate. Another marker that operates similarly to the met3 gene can be, for example, the niaA gene encoding nitrate reductase, which can be used in the selection/counterselection scheme described in this embodiment. For the niaA gene, the filamentous fungal host cells can be niaA+, whereby correct integration of the second construct generates niaA− progeny which are resistant to chlorate used during counterselection. In one embodiment, confirmation of correct integration of the first and/or second construct into the protoplast genome is confirmed by sequencing the genome of the protoplast using such as, for example next generation sequencing (NGS).

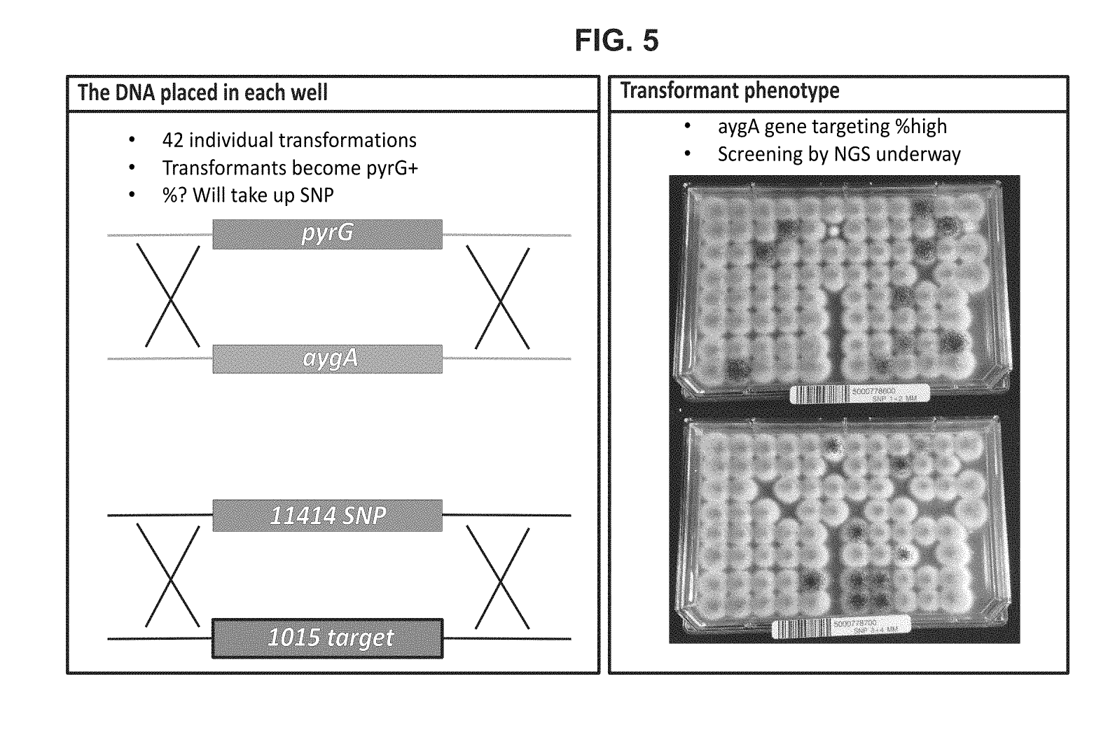

An example of the embodiment where the first construct comprises a sequence for an endogenous gene and the target gene to be removed or inactivated is the copy of the endogenous gene present in the genome of the protoplast prior to transformation is shown in FIG. 5. In this example, the first construct comprises sequence for an endogenous gene involved in citric acid production in filamentous fungus that comprises a SNP that is integrated into the locus for said endogenous gene lacking said SNP, while the second construct comprises sequence for the auxotrophic marker gene pyrG that is integrated into the locus for the colorimetric marker gene aygA. In this example, the filamentous fungal host cell is pyrG negative or uracil auxotrophic. Accordingly, purification of homokaryotic protoplast transformants entails growing said transformants on minimal media lacking uracil. As shown in FIG. 5, homokaryotic transformants will not only be uracil prototrophs, but will also be pure yellow in color, indicting incorporation of the pyrG gene and removal of the aygA gene. In one embodiment, confirmation of correct integration of the first and/or second construct into the protoplast genome is confirmed by sequencing the genome of the protoplast using such as, for example next generation sequencing (NGS).

In one embodiment, the second construct comprises an expression cassette that encodes a recyclable or reversible marker. The recyclable or reversible marker can be a disruption neo-pyrG-neo expression cassette. The neo-pyrG-neo construct can be co-transformed with the first construct as described in the above embodiments in a ura− strain of filamentous fungal host cell (e.g., A. niger) and homokaryotic transformants can be selected by plating on uracil deficient medium and selecting pure yellow uracil prototrophs as described above. Subsequently, use of pyrG selection can be regenerated by plating said homokaryotic transformants on 5-FOA containing medium and selecting transformants that grow on said 5-FOA medium, which indicates that said transformants have undergone an intrachromosomal recombination between the neo repeats that results in excision of the pyrG gene.

Library Generation

A further aspect of the disclosure can include the construction and screening of fungal mutant libraries, and fungal mutant libraries prepared by the methods disclosed herein. The libraries may be obtained by transformation of the fungal hosts according to this disclosure with any means of integrative transformation, using methods known to those skilled in the art. A library of fungi based on the preferred host strains generated using the methods and systems provided herein may be handled and screened for desired properties or activities of exogenous proteins in miniaturized and/or high-throughput format screening methods. Property or activity of interest can mean any physical, physicochemical, chemical, biological, or catalytic property, or any improvement, increase, or decrease in such a property, associated with an exogenous protein of a library member. The library may also be screened for metabolites, or for a property or activity associated with a metabolite, produced as a result of the presence of exogenous and/or endogenous proteins. The library may also be screened for fungi producing increased or decreased quantities of such protein or metabolites.

In one embodiment, the methods and systems provided herein generate a plurality of protoplasts such that each protoplast from the plurality of protoplasts is transformed with a single first construct from a plurality of first constructs and a single second construct from a plurality of second constructs. Further to this embodiment, a first polynucleotide in each first construct from the plurality of first constructs comprises a different mutation and/or genetic control or regulatory element while a second polynucleotide in each second construct from the plurality of second constructs is identical. The method further comprises transforming and purifying homokaryotic transformants using selection/counterselection as described herein two or more time in order to generate a library of filamentous fungal cells such that each filamentous fungal cell in the library comprises a first polynucleotide with a different mutation and/or genetic control or regulatory element. In one embodiment, the first polynucleotide comprises sequence for a target filamentous fungal gene or a heterologous gene comprising a mutation such that the iterative process generates a library of filamentous fungal cells upon regeneration of the protoplasts such that each member of the library comprises a target filamentous fungal gene or a heterologous gene with a distinct mutation. In one embodiment, the mutation is a SNP and the methods thereby produces a SNPSwap library. In one embodiment, the target filamentous fungal gene is a gene involved in citric acid production and the plurality of first constructs is the library of SNPs provided in Table 1. In another embodiment, the first polynucleotide comprises sequence for a target filamentous fungal gene or a heterologous gene operably linked to a genetic control or regulatory element such that the iterative process described herein generates a library of filamentous fungal cells upon regeneration of the protoplasts such that each member of the library comprises a target filamentous fungal gene or a heterologous gene operably linked to a distinct genetic control or regulatory element. In one embodiment, the genetic control or regulatory element is a promoter and the methods thereby produces a Promoter or PRO library. In one embodiment, the genetic control or regulatory element is a terminator and the methods thereby produces a Terminator or STOP library. The promoter and/or terminator sequence can be an promoter or terminator sequence provided herein and/or known in the art for expression in a filamentous fungal host cells used in the methods and systems provided herein.

TABLE 1
SNPs potentially involved in citric acid production in A. niger.

| | | Sequence | | |
|---|---|---|---|---|
| Mutation name | Location | change | orientation | Contig |
| FungiSNP_01 | 50669-680224 | ~ > ~ | 680224 | chr_1_1 |
| FungiSNP_02 | 1172974 | G > A | + | chr_1_1 |
| FungiSNP_03 | 367948 | C > T | + | chr_1_2 |
| FungiSNP_04 | 549014 | C > G | − | chr_1_2 |
| FungiSNP_05 | 1330718 | G > A | + | chr_1_2 |
| FungiSNP_06 | 662258 | G> | + | chr_2_1 |
| FungiSNP_07 | 673547 | G > A | − | chr_2_1 |
| FungiSNP_08 | 946654 | T> | + | chr_2_1 |
| FungiSNP_09 | 641661 | T > A | − | chr_2_2 |
| FungiSNP_10 | 2316591 | G > A | + | chr_2_2 |
| FungiSNP_11 | 935908 | A > G | − | chr_3_1 |
| FungiSNP_12 | 205638 | T > A | + | chr_3_2 |
| FungiSNP_13 | 268107 | T > C | + | chr_3_3 |
| FungiSNP_14 | 186943 | A > T | + | chr_3_4 |
| FungiSNP_15 | 276232 | C > T | + | chr_3_4 |
| FungiSNP_16 | 1287891 | T > C | − | chr_4_1 |
| FungiSNP_17 | 1639965 | A > T | + | chr_4_1 |
| FungiSNP_18 | 1826343 | G > A | − | chr_4_1 |
| FungiSNP_19 | 1358794 | C > A | + | chr_4_2 |
| FungiSNP_20 | 1466380 | CTA> | + | chr_4_2 |
| FungiSNP_21 | 1700330 | C > A | − | chr_4_2 |
| FungiSNP_22 | 2873296 | A > G | + | chr_4_2 |
| FungiSNP_23 | 815022 | G > A | + | chr_5_2 |
| FungiSNP_24 | 831672 | G > A | − | chr_5_2 |
| FungiSNP_25 | 1507652 | >A | + | chr_5_2 |
| FungiSNP_26 | 442488 | T > C | + | chr_6_1 |
| FungiSNP_27 | 93202-103239 | ~ > ~ | + | chr_6_2 |
| FungiSNP_28 | 972833 | A > T | + | chr_6_2 |
| FungiSNP_29 | 972932 | A> | + | chr_6_2 |
| FungiSNP_30 | 1183094 | G> | + | chr_6_2 |
| FungiSNP_31 | 1701762 | T > G | + | chr_6_2 |
| FungiSNP_32 | 236406 | G > A | − | chr_7_1 |
| FungiSNP_33 | 2350056 | A> | + | chr_7_1 |
| FungiSNP_34 | 375013 | C > T | + | chr_8_1 |
| FungiSNP_35 | 1272037 | C > T | + | chr_8_1 |
| FungiSNP_36 | 281612 | T > C | + | chr_8_2 |
| FungiSNP_37 | 565087 | A > G | + | chr_8_2 |
| FungiSNP_38 | 865958 | A> | + | chr_8_2 |
| FungiSNP_39 | 947633 | A> | + | chr_8_2 |
| FungiSNP_40 | 2482267 | G > A | + | chr_8_2 |
| FungiSNP_41 | 2486601 | G> | + | chr_8_2 |
| FungiSNP_42 | 2709491 | T > C | + | chr_8_2 |
| FungiSNP_43 | 2708043 | >A | ~ | chr_8_2 |

SNP Swapping

In one embodiment, the methods and systems provided herein are utilized for SNP swapping in order to generate filamentous fungal libraries comprising filamentous fungal with individual SNPs or combinations of SNPs.

The resultant microbes that are engineered via this process form HTP genetic design libraries.

The HTP genetic design library can refer to the actual physical microbial strain collection that is formed via this process, with each member strain being representative of the presence or absence of a given SNP, in an otherwise identical genetic background, said library being termed a “SNP swap microbial strain library.”

Furthermore, the HTP genetic design library can refer to the collection of genetic perturbations—in this case a given SNP being present or a given SNP being absent—said collection being termed a “SNP swap library.”

Thus, SNP swapping involves the reconstruction of host organisms with optimal combinations of target SNP “building blocks” with identified beneficial performance effects. Thus, in some embodiments, SNP swapping involves consolidating multiple beneficial mutations into a single strain background, either one at a time in an iterative process, or as multiple changes in a single step. Multiple changes can be either a specific set of defined changes or a partly randomized, combinatorial library of mutations.

In other embodiments, SNP swapping also involves removing multiple mutations identified as detrimental from a strain, either one at a time in an iterative process, or as multiple changes in a single step. Multiple changes can be either a specific set of defined changes or a partly randomized, combinatorial library of mutations. In some embodiments, the SNP swapping methods of the present disclosure include both the addition of beneficial SNPs, and removing detrimental and/or neutral mutations.

In some embodiments, the SNP swapping methods of the present disclosure comprise the step of introducing one or more SNPs identified in a mutated strain (e.g., a strain from amongst S₂₋ₙGen₂₋ₙ) to a reference strain (S₁Gen₁) or wild-type strain.

In other embodiments, the SNP swapping methods of the present disclosure comprise the step of removing one or more SNPs identified in a mutated strain (e.g., a strain from amongst S₂₋ₙGen₂₋ₙ).

In some embodiments, each generated strain comprising one or more SNP changes (either introducing or removing) is cultured and analyzed under one or more criteria of the present disclosure (e.g., production of a chemical or product of interest). Data from each of the analyzed host strains is associated, or correlated, with the particular SNP, or group of SNPs present in the host strain, and is recorded for future use. Thus, the present disclosure enables the creation of large and highly annotated HTP genetic design microbial strain libraries that are able to identify the effect of a given SNP on any number of microbial genetic or phenotypic traits of interest. The information stored in these HTP genetic design libraries informs the machine learning algorithms of the HTP genomic engineering platform and directs future iterations of the process, which ultimately leads to evolved microbial organisms that possess highly desirable properties/traits.

HTP Automated Systems

In some embodiments, the methods and systems provided herein comprise automated steps. For example, the generation of protoplasts, transformation of protoplasts and/or purifying homokaryotic protoplasts via selection/counterselection as described herein can be automated. As described herein, the methods and system can contain a further step of screening purified homokaryotic transformants for the production of a protein or metabolite of interest. The automated methods of the disclosure can comprise a robotic system. The systems outlined herein can be generally directed to the use of 96- or 384-well microtiter plates, but as will be appreciated by those in the art, any number of different plates or configurations may be used. In addition, any or all of the steps outlined herein may be automated; thus, for example, the systems may be completely or partially automated. The automated methods and systems can be high-throughput. For purposes of this disclosure, high-throughput screening can refers to any partially- or fully-automated method that is capable of evaluating about 1,000 or more transformants per day, and particularly to those methods capable of evaluating 5,000 or more transformants per day, and most particularly to methods capable of evaluating 10,000 or more transformants per day.

As described herein, the methods and system provided herein can comprise a screening step such that a transformant generated and purified as described herein is screened or tested for the production of a product of interest. The product of interest can be any product of interest provided herein such as, for example, an alcohol, pharmaceutical, metabolite, protein, enzyme, amino acid, or acid (e.g., citric acid). Accordingly, the methods and systems provided herein can further comprise culturing a clonal colony or culture purified according to the methods of the disclosure, under conditions permitting expression and secretion of the product of interest and recovering the subsequently produced product of interest. As described herein, the product of interest can an exogenous and/or heterologous protein or a metabolite produced as the result of the expression of an exogenous and or heterologous protein.

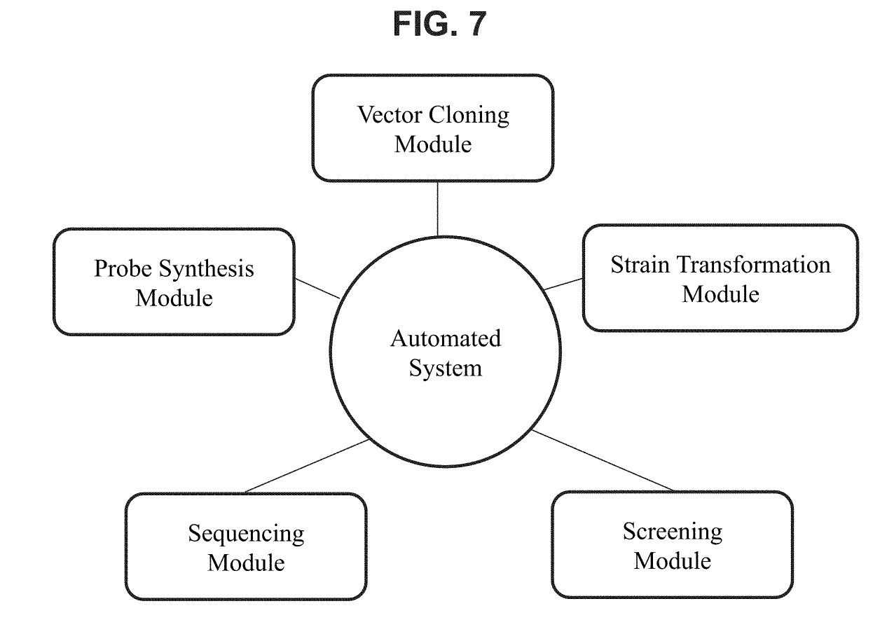

In some embodiments, the automated systems of the present disclosure comprise one or more work modules. For example, in some embodiments, the automated system of the present disclosure comprises a DNA synthesis module, a vector cloning module, a strain transformation module, a screening module, and a sequencing module (see FIG. 7).

As will be appreciated by those in the art, an automated system can include a wide variety of components, including, but not limited to: liquid handlers; one or more robotic arms; plate handlers for the positioning of microplates; plate sealers, plate piercers, automated lid handlers to remove and replace lids for wells on non-cross contamination plates; disposable tip assemblies for sample distribution with disposable tips; washable tip assemblies for sample distribution; 96 well loading blocks; integrated thermal cyclers; cooled reagent racks; microtiter plate pipette positions (optionally cooled); stacking towers for plates and tips; magnetic bead processing stations; filtrations systems; plate shakers; barcode readers and applicators; and computer systems.

In some embodiments, the robotic systems of the present disclosure include automated liquid and particle handling enabling high-throughput pipetting to perform all the steps in the process of gene targeting and recombination applications. This includes liquid and particle manipulations such as aspiration, dispensing, mixing, diluting, washing, accurate volumetric transfers; retrieving and discarding of pipette tips; and repetitive pipetting of identical volumes for multiple deliveries from a single sample aspiration. These manipulations are cross-contamination-free liquid, particle, cell, and organism transfers. The instruments perform automated replication of microplate samples to filters, membranes, and/or daughter plates, high-density transfers, full-plate serial dilutions, and high capacity operation.

The automated system can be any known automated high-throughput system known in the art. For example, the automated system can be the automated microorganism handling tool is described in Japanese patent application publication number 11-304666. This device is capable of the transfer of microdroplets containing individual cells, and it is anticipated that the fungal strains of the present disclosure, by virtue of their morphology, will be amenable to micromanipulation of individual clones with this device. An additional example of an automated system for use in the methods and system of the present disclosure is the automated microbiological high-throughput screening system described in Beydon et al., J. Biomol. Screening 5:13 21 (2000). The automated system for use herein can be a customized automated liquid handling system. In one embodiment, the customized automated liquid handling system of the disclosure is a TECAN machine (e.g. a customized TECAN Freedom Evo).

In some embodiments, the automated systems of the present disclosure are compatible with platforms for multi-well plates, deep-well plates, square well plates, reagent troughs, test tubes, mini tubes, microfuge tubes, cryovials, filters, micro array chips, optic fibers, beads, agarose and acrylamide gels, and other solid-phase matrices or platforms are accommodated on an upgradeable modular deck. In some embodiments, the automated systems of the present disclosure contain at least one modular deck for multi-position work surfaces for placing source and output samples, reagents, sample and reagent dilution, assay plates, sample and reagent reservoirs, pipette tips, and an active tip-washing station.

In some embodiments, the automated systems of the present disclosure include high-throughput electroporation systems for transforming the protoplasts. In some embodiments, the high-throughput electroporation systems are capable of transforming cells in 96 or 384-well plates. In some embodiments, the high-throughput electroporation systems include VWR® High-throughput Electroporation Systems, BTX™, Bio-Rad® Gene Pulser MXcell™ or other multi-well electroporation system.

In some embodiments, the automated systems comprise an integrated thermal cycler and/or thermal regulators that are used for stabilizing the temperature of heat exchangers such as controlled blocks or platforms to provide accurate temperature control of incubating samples from 0° C. to 100° C.

In some embodiments, the automated systems of the present disclosure are compatible with interchangeable machine-heads (single or multi-channel) with single or multiple magnetic probes, affinity probes, replicators or pipetters, capable of robotically manipulating liquid, particles, cells, and multi-cellular organisms. Multi-well or multi-tube magnetic separators and filtration stations manipulate liquid, particles, cells, and organisms in single or multiple sample formats.

In some embodiments, the automated systems of the present disclosure are compatible with camera vision and/or spectrometer systems. Thus, in some embodiments, the automated systems of the present disclosure are capable of detecting and logging color and absorption changes in ongoing cellular cultures.

In some embodiments, the automated system of the present disclosure is designed to be flexible and adaptable with multiple hardware add-ons to allow the system to carry out multiple applications. The automated system for use in the methods provided herein can comprise software program modules. The software program modules can allow creation, modification, and running of methods. The systems can further comprise diagnostic modules. The diagnostic modules can allow setup, instrument alignment, and motor operations. The systems can still further comprise customized tools, labware, liquid and particle transfer patterns and/or a database(s). The customized tools, labware, and liquid and particle transfer patterns can allow different applications to be programmed and performed. The database can allow method and parameter storage. Further, robotic and computer interfaces present in the system can allow communication between instruments.

Persons having skill in the art will recognize the various robotic platforms capable of carrying out the HTP methods of the present disclosure.

Computer System Hardware

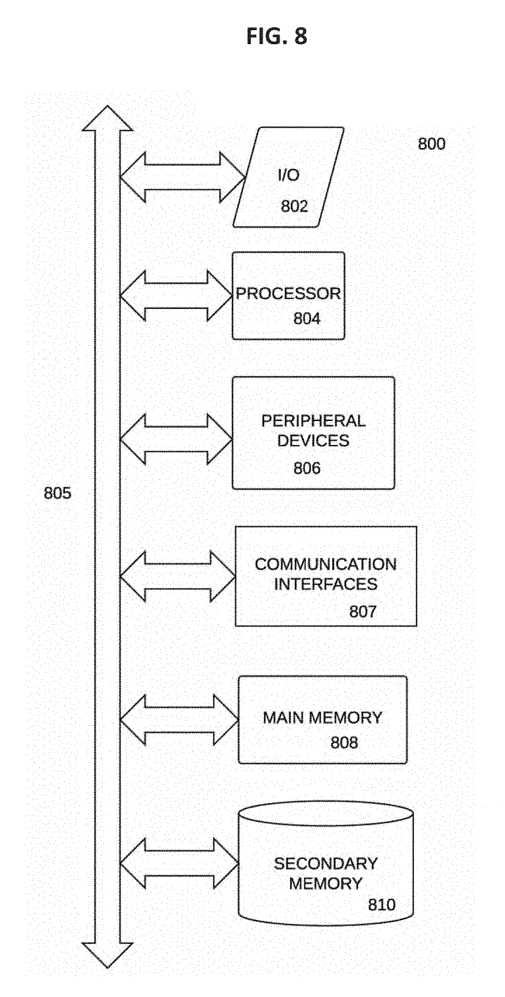

FIG. 8 illustrates an example of a computer system 800 that may be used to execute program code stored in a non-transitory computer readable medium (e.g., memory) in accordance with embodiments of the disclosure. The computer system includes an input/output subsystem 802, which may be used to interface with human users and/or other computer systems depending upon the application. The I/O subsystem 802 may include, e.g., a keyboard, mouse, graphical user interface, touchscreen, or other interfaces for input, and, e.g., an LED or other flat screen display, or other interfaces for output, including application program interfaces (APIs). Other elements of embodiments of the disclosure, such as the components of the LIMS system, may be implemented with a computer system like that of computer system 800.

Program code may be stored in non-transitory media such as persistent storage in secondary memory 810 or main memory 808 or both. Main memory 808 may include volatile memory such as random access memory (RAM) or non-volatile memory such as read only memory (ROM), as well as different levels of cache memory for faster access to instructions and data. Secondary memory may include persistent storage such as solid state drives, hard disk drives or optical disks. One or more processors 804 reads program code from one or more non-transitory media and executes the code to enable the computer system to accomplish the methods performed by the embodiments herein. Those skilled in the art will understand that the processor(s) may ingest source code, and interpret or compile the source code into machine code that is understandable at the hardware gate level of the processor(s) 804. The processor(s) 804 may include graphics processing units (GPUs) for handling computationally intensive tasks. Particularly in machine learning, one or more CPUs 804 may offload the processing of large quantities of data to one or more GPUs 804.

The processor(s) 804 may communicate with external networks via one or more communications interfaces 807, such as a network interface card, WiFi transceiver, etc. A bus 805 communicatively couples the I/O subsystem 802, the processor(s) 804, peripheral devices 806, communications interfaces 807, memory 808, and persistent storage 810. Embodiments of the disclosure are not limited to this representative architecture. Alternative embodiments may employ different arrangements and types of components, e.g., separate buses for input-output components and memory subsystems.

Those skilled in the art will understand that some or all of the elements of embodiments of the disclosure, and their accompanying operations, may be implemented wholly or partially by one or more computer systems including one or more processors and one or more memory systems like those of computer system 800. In particular, any robotics and other automated systems or devices described herein may be computer-implemented. Some elements and functionality may be implemented locally and others may be implemented in a distributed fashion over a network through different servers, e.g., in client-server fashion, for example. In particular, server-side operations may be made available to multiple clients in a software as a service (SaaS) fashion.

The term component in this context refers broadly to software, hardware, or firmware (or any combination thereof) component. Components are typically functional components that can generate useful data or other output using specified input(s). A component may or may not be self-contained. An application program (also called an “application”) may include one or more components, or a component can include one or more application programs.

Some embodiments include some, all, or none of the components along with other modules or application components. Still yet, various embodiments may incorporate two or more of these components into a single module and/or associate a portion of the functionality of one or more of these components with a different component.

The term “memory” can be any device or mechanism used for storing information. In accordance with some embodiments of the present disclosure, memory is intended to encompass any type of, but is not limited to: volatile memory, nonvolatile memory, and dynamic memory. For example, memory can be random access memory, memory storage devices, optical memory devices, magnetic media, floppy disks, magnetic tapes, hard drives, SIMMs, SDRAM, DIMMs, RDRAM, DDR RAM, SODIMMS, erasable programmable read-only memories (EPROMs), electrically erasable programmable read-only memories (EEPROMs), compact disks, DVDs, and/or the like. In accordance with some embodiments, memory may include one or more disk drives, flash drives, databases, local cache memories, processor cache memories, relational databases, flat databases, servers, cloud based platforms, and/or the like. In addition, those of ordinary skill in the art will appreciate many additional devices and techniques for storing information can be used as memory.

Memory may be used to store instructions for running one or more applications or modules on a processor. For example, memory could be used in some embodiments to house all or some of the instructions needed to execute the functionality of one or more of the modules and/or applications disclosed in this application.

### EXAMPLES

### Example 1: HTP Genomic Engineering of Filamentous Fungi: Generation & Storage of Filamentous Fungal Protoplasts

Generation of Protoplasts

As shown in FIG. 1A, 100 milliliters of complete media was inoculated with 10⁶ conidia/ml of Aspergillus niger and grown overnight at 150 rpm at 30° C. Following the overnight growth. the mycelia were harvested by filtering the culture through Miracloth. Subsequently, the mycelia were rinsed thoroughly with sterile water. For the experiments described in the following examples, two strains of A. niger were used, A. Niger strain 1015 and A. niger strain 11414. Harvested and washed mycelia were then subjected to enzymatic digestion by with a VinoTaste Pro (VTP) enzymatic cocktail.

For A. niger strain 1015, enzymatic digestion was performed by first making 50 ml of 60 mg/ml of VTP in protoplasting buffer (1.2M magnesium sulfate, 50 mM phosphate buffer, pH 5). After dissolving the VTP, the buffer was placed in clean Oakridge tubes and spun at 15,000×g for 15 minutes. The solution was then filter sterilized after centrifugation. Once made, some of the harvested mycelia was added to the VTP solution and the mycelia was digested at 30° C. at 80 rpm for 2-4 hours. At various intervals during VTP digestion, small samples were examined under 400× magnification for the presence of protoplasts (i.e., large round cells that are larger than conidia and are sensitive to osmotic lysis). When most or all of the mycelia were digested, the culture was filtered through sterile Miracloth such that 25 ml of the flow through containing the protoplasts were separated into 1 of 2 50 ml Falcon tubes. To each of the 25 ml samples, 5 ml of 0.4M ST buffer (0.4M Sorbitol, 100 mM Tris, pH 8) was gently overlaid. The overlaid samples were then spun at 800×g for 15 minutes at 4° C. in order to form a visible layer between the ST and digestion buffers. The protoplasts were then removed with a pipette and mixed gently with 25 ml of ST solution (1.0 M sorbitol, 50 mM Tris, Ph 8.0) and respun at 800×g for 10 minutes. The protoplasts should pellet at the bottom of the tube. The protoplasts were then resuspended in 25 ml of ST solution and collected by centrifugation at 800×g for 10 minutes.

For A. niger strain 11414, enzymatic digestion was performed by first making 40 ml of 30 mg/ml of VTP in protoplasting buffer (0.6M ammonium sulfate, 50 mM Maleic Acid, pH 5.5). All of the harvested mycelia were added to the VTP solution and the mycelia were digested at 30° C. at 70 rpm for 3˜4 hours. At various intervals during VTP digestion, small samples were examined under 400× magnification for the presence of protoplasts. When most or all of the mycelia were digested, the culture was filtered through sterile Miracloth. The filtrate was then spun at 800×g for 10 min at 4° C. to pellet the cells. 25 ml of ST solution (1.0M sorbitol, 50 mM Tris, pH 8.0) was added and the cells were resuspended and respun. The cells were then washed in 10 ml of STC buffer (1.0M sorbitol, 50 mM Tris, pH 8.0, 50 mM CaCl₂)) and collected by centrifugation at 800×g for 10 min. The protoplasts (˜10⁸/ml) were counted and adjusted to be at 1.2×10⁷/ml.

For protoplasts generated from either A. niger strain (i.e., 1015 or 11414), following enzymatic digestion, 20% v/v of a 40% PEG solution (40% PEG-4000 in STC buffer)) was added to the protoplasts and mixed gently followed by adding 7% v/v of dimethyl sulfoxide (DMSO) to make a8% PEG/7% DMSO solution. Following resuspension, the protoplasts were distributed to 96 well (25-50 microliters) microtiter plates using an automated liquid handler as depicted in FIG. 1, followed by storage at at least −80° C. prior to transformation.

### Example 2: HTP Genomic Engineering of Filamentous Fungi: Demonstration of Co-Transformation of Filamentous Fungal Protoplasts-Proof of Principle

Preparation of Targeting DNA

In an effort to provide proof of concept (POC) for the automated filamentous fungal transformation and screening method depicted in FIG. 1A, the DNA sequence of the Aspergillus niger argB gene was obtained and the proper reading frame was determined. A set of SNPs were then designed such that integration of any of said SNPs into the argB locus of the A. niger genome would result in null mutation of the argB gene. The designs were generated as in silico constructs that predicted a set of oligomers that were used to build the constructs using Gibson assembly.

Automated Transformation of Protoplasts

Protoplasts derived from A. niger strain 1015 generated and stored in 96 well plates (100 microliters protoplast/well) as described in Example 1 were then subjected to traditional PEG Calcium mediated transformations using automated liquid handlers to combine the SNP DNA constructs with the protoplast-PEG/DMSO mixtures in the 96 well plates. More specifically, to 100 microliters of protoplasts, 1-10 micrograms of the SNP DNA constructs (in a volume of 10 microliters or less) were added and the mixture was incubated on ice for 15 minutes. To this mixture, 1 ml of 40% PEG was added and incubated for 15 minutes for room temperature. Subsequently, 10 ml of minimum medium plus 1M sorbitol was added and shaken at 80 rpm for 1 hour at 30° C. Following this incubation, the protoplasts were spun down at 800×g for 5 minutes and then resuspended in 12 ml of minimal medium containing 1M sorbitol and 0.8% agar. The following day, using an additional automated liquid handling step, the protoplasts were plated on to selective media (i.e., minimal media+arginine) and non-selective media (i.e., minimal media). Successful transformation of the protoplasts generated with the automated transformation protocol would be expected to be auxotrophic for arginine and thus not grow on minimal media lacking arginine due to targeting of the argB gene by the SNP constructs.

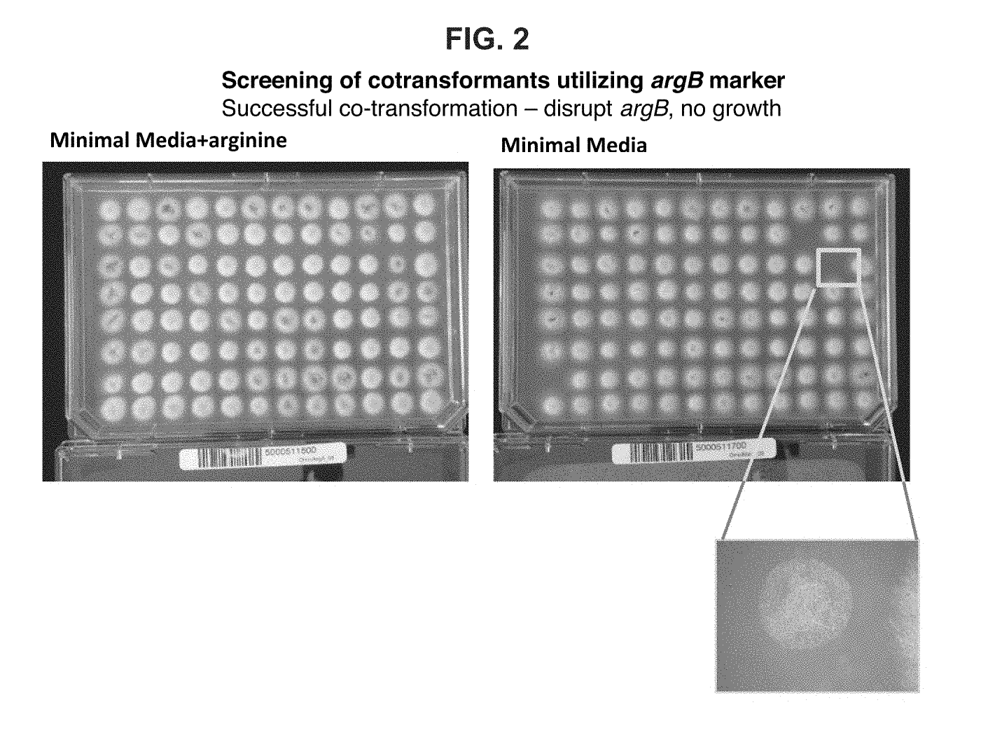

As shown in FIG. 2, about 3% of the transformants displayed integration of the targeting DNA constructs at the correct (i.e., argB) locus as evidenced by lack of growth in the minimal media lacking arginine. Confirmation of integration of the SNP containing constructs at the correct locus will be confirmed via next generation sequencing.

### Example 3: HTP Genomic Engineering of Filamentous Fungi: Demonstration of Co-Transformation of Filamentous Fungal Protoplasts-Proof of Principle Using Colorimetric Selection/Counterselection

This example demonstrates an additional proof of principle for the automated, HTP co-transformation of filamentous fungal cells and further demonstrates the use of selection/counterselection for the isolation of desired transformants.

### Aspergillus Niger Protoplast Formation and Transformation

A large volume (500 ml) of protoplasts of a eukaryotic fungal strain of Aspergillus niger, ATCC 1015, was generated using a commercially available enzyme mixture which contains beta-glucanase activity as described in Example 1. The protoplasts were isolated from the enzyme mixture by centrifugation and were ultimately re-suspended in a buffer containing calcium chloride by the method described in Example 1.

The protoplasts were aliquoted and frozen at negative 80 degrees Celsius in containers containing a suspension of dimethyl sulfoxide and polyethylene glycol (PEG) as described in Example 1. In some embodiments, the present disclosure teaches that a stock of 96-well microtiter plates containing 25-50 microliters of protoplasts in each well can be prepared and frozen in large batches for large scale genome editing campaigns using this technique.

Traditional PEG Calcium mediated transformations were carried out by automated liquid handlers, which combined the DNA with the protoplast-PEG mixtures in the 96 wells. An additional automated liquid handling step was used to plate the transformation on to selective media after transformation.

### Automated Screening of Transformants

As discussed in more detail below, the A. niger cells used in this example lacked a functional pyrG gene (i.e., pyrG−) were transformed with a functional pyrG gene, which permitted transformed cells to grow in the absence of Uracil. As shown in FIG. 4A-B, the pyrG gene of this example was further designed to incorporate into the location of A. niger's wild type met3 gene, thus incorporating a disruption to the naturally occurring met3 gene. Disruption of the met3 gene further results in the transformants being methionine auxotrophs, providing a secondary screening method for identifying transformants.

Transformants grown on the selective media without Uracil were isolated and placed into individual wells of a second microtiter plate. The transformants in the second microtiter plate were allowed to grow and sporulate for 2-3 days, before being resuspended in a liquid consisting of water and a small amount of detergent to generate a spore stock suitable for storage and downstream automated screening.

A small aliquot of each of the aforementioned spore stocks was then used to inoculate liquid media in a third 96 well PCR plate. These small cultures are allowed to grow over night in a stationary incubator so that the yellow-pigment containing spores germinate and form hyphae that are more amenable to selection, and downstream steps.

Following the culturing step, the hyphae of the third PCR plate were lysed by adding a commercially available buffer and heating the cultures to 99 degrees Celsius for 20 minutes. The plates were then centrifuged to separate the DNA suspension supernatant from the cell/organelle pellets. The DNA extractions were then used for PCR analysis to identify cell lines comprising the desired DNA modifications.

### Co-Transformation for Integration of SNPs-Design of SNPs

The DNA sequence of the Aspergillus niger gene aygA was obtained and the proper reading frame was determined. Four distinct types of mutations were designed, which if integrated would result in a null mutation.

The mutations included a single base pair change that incorporates an in-frame stop codon, a small two base pair deletion, a three-base pair integration, and a larger 100 base pair deletion all of which if properly integrated will eliminate aygA activity. Strains lacking aygA activity have a yellow spore phenotype. The designs were generated as in silico constructs that predicted a set of oligomers that were used to build the constructs using Gibson assembly.

### Integration of SNPs by Co-Transformation

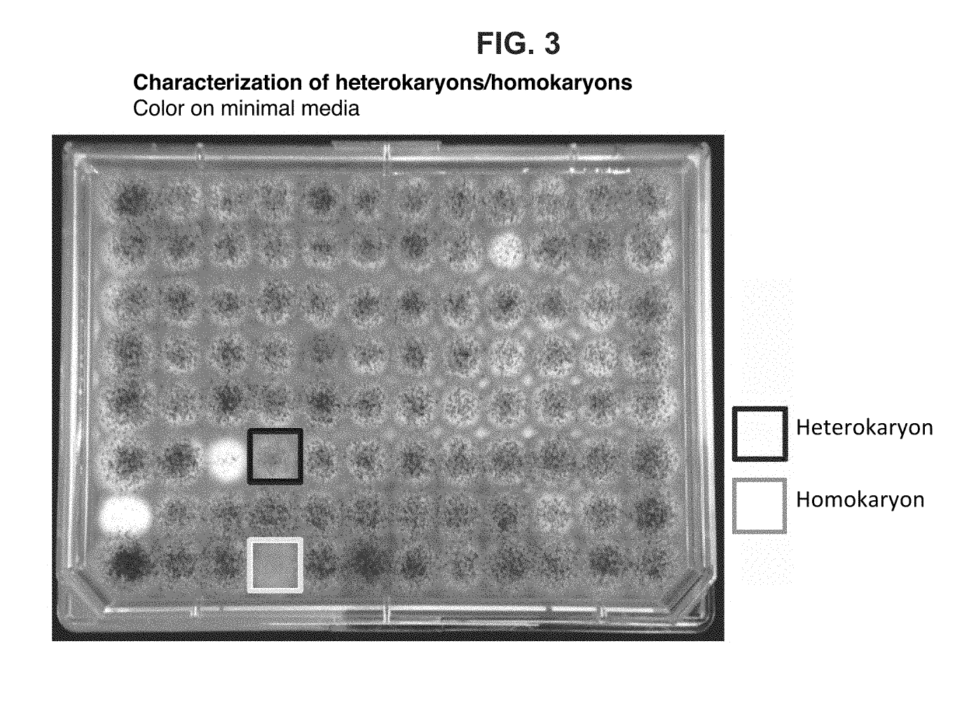

Using the transformation approach described above, amplicons containing the small changes were incorporated into the genome of an Aspergillus niger strain 1015. As previously discussed, this strain of Aspergillus niger comprised a non-functional pyrG gene, and was therefore unable to grow in the absence of exogenous uracil. Cells that had successfully integrated the pyrG gene were now capable of growth in the absence of uracil. Of these pyrG+transformants, isolates that also integrated the small mutations in the aygA gene exhibited the yellow spore phenotype. (see FIGS. 3 and 4A). The presence of the mutation was also detected through sequencing of small amplicons that contain the region targeted for the SNP exchange (FIG. 4B).

### Example 4: HTP Genomic Engineering of Filamentous Fungi: Implementation of an HTP SNP Library Strain Improvement Program to Improve Citric Acid Production in Eukaryote Aspergillus niger ATCC 11414

Example 3 above described the techniques for automating the genetic engineering techniques of the present disclosure in a high throughput manner. This example applies the techniques described above to the specific HTP strain improvement of Aspergillus niger strain ATCC11414.

Aspergillus niger is a species of filamentous fungi used for the large scale production of citric acid through fermentation. Multiple strains of this species have been isolated and shown to have varying capacity for production of citric and other organic acids. The HTP strain engineering methods of the present disclosure can be used to combine causative alleles and eliminate detrimental alleles to improve citric acid production.

### Genetic Engineering Using a Split-Marker Approach

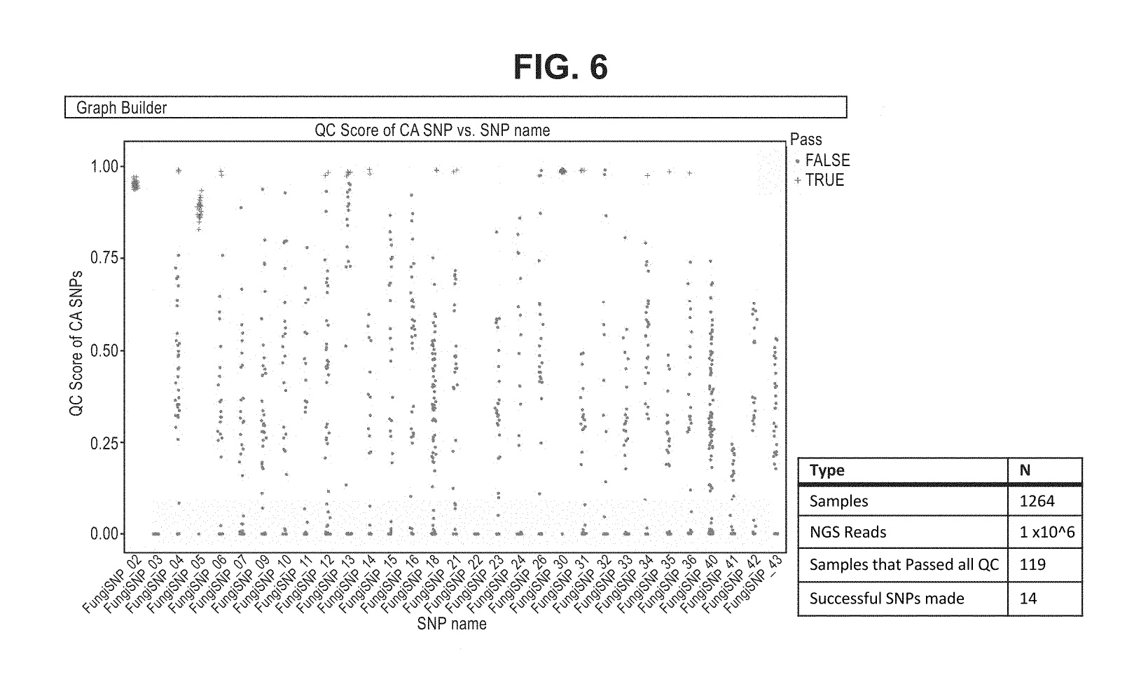

The initial steps of the split marker mediated SNP exchange are the same as in Example 3 and are represented in FIG. 1D as steps 1 and 2. In step 2 the transforming DNA consists of two separate linear fragments that contain non-complementing halves of the marker fused to homologous DNA for targeting the SNP to the proper locus. The transformations are placed onto selective media and allowed to grow. Properly complemented strains that have stable integration of the DNA will form colonies. These colonies are picked either by hand or by an automated platform to individual wells in a microtiter plate which contains 100-200 microliters of selective agar media. The picked transformants are allowed to grow and propagate spores as indicated in step 4. The spores of Aspergillus niger are uninucleate and are inherently clonal. The transformed strains are purified to homokaryon (all nuclei in the cell are of identical genotype) by taking small numbers of spores and plating them again onto selective media. This process is represented by arrows in FIG. 1D. Repeated reduction of the population to small numbers of clonal pores will result in a homokaryon in each well. These purified strains in wells are then plated to media containing a counterselection agent that is toxic to strains that contain the selectable marker. Strains that have taken up the marker that is flanked by the direct repeats containing the SNP will lose the marker at a frequency that is directly correlated to the size of the direct repeats. For example, a 1,000 base pair direct repeat is less stable than a 100 base pair direct repeat. This loop out phase is step 6 in FIG. 1D. The looped out strains are then screened in a manner similar to that done in example 2 using NGS. FIG. 4C contains data from a SNPSWP campaign that was performed utilizing split marker integration an loop out. In this example over 1200 individual looped out samples were screened using NGS. From this set 119 successful strains were generated and appear as red dots in the upper left corner of FIG. 4C because 100% of the amplicons from that well contain the SNP from the production strain. The samples designated in blue contain both amplicons with the desired SNP and the native base pair at this locus. These strains may be made homokayotic using the spore propagation and passaging represented in steps 4 and 5 in FIG. 1D. The 119 samples that have passed QC can be analyzed for their impact on desired strain improvement traits. The success rate of SNP introduction across various SNP positions is shown in FIG. 6.

Identification of a Library of Genetic Design Library for SNPs from Natural A. niger Strain Variants.

A. niger strain ATCC 1015 was identified as a producer of citric acid in the early twentieth century. An isolate of this strain named ATCC 11414, was later found to exhibit increased citric acid yield over its parent. For example, A. niger strain ATCC 1015 on average produces 7 grams of citric acid from 140 grams of glucose in media containing ammonium nitrate, but lacking both iron and manganese cations. Isolate strain ATCC 11414 on the other hand, exhibits a 10-fold yield increase (70 grams of citric acid) under the same conditions. Moreover, strain ATCC 11414 spores germinate and grow better in citric acid production media than do spores of strain 1015.

In order to identify potential genetic sources for these phenotypic differences, the genomes of both the ATCC 1015 and ATCC 11414 strains were sequenced and analyzed. The resulting analysis identified 42 SNPs distinguishing the 1015 and 11414 strains.

### Exchanging Causative Alleles

Protoplasts were prepared from strain ATCC 1015 (“base strain”) for transformation as described in Example 1. Each of the above-identified 42 SNPs were then individually introduced into the base strain via the gene editing techniques of the present disclosure (see FIG. 5). Each SNP was co-transformed with the functional pyrG and aygA gene mutation as described above. Transformants that had successful gene targeting to the aygA locus produced yellow spores (FIG. 5).

### Screening for Successful Integration

Transformants containing putative SNPs were isolated and a spore stock was propagated as stated above. Amplicons that contain the region of DNA containing the putative SNP were analyzed by next generation sequencing. Using this approach it is possible to determine successful integration events within each transformant even in the presence of the parental DNA. This capability is essential to determine targeting in fungi which can grow as heterokaryons which contain nuclei with differing genotype in the same cell.

Transformants were further validated for presence of the desired SNP change. The co-transformants that had the yellow spore phenotype also contained proper integration of the citric acid SNP in approximately 30% of the isolates

Next, the created SNP swap microbial strain library will be phenotypically screened in order to identify SNPs beneficial to the production of citric acid. The information will be utilized in the context of the HTP methods of genomic engineering described herein, to derive an A. niger strain with increased citric acid production

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as an acknowledgment or any form of suggestion that they constitute valid prior art or form part of the common general knowledge in any country in the world.

1. A method for producing a filamentous fungal strain, the method comprising:
a.) transforming a plurality of protoplasts prepared from a culture of filamentous fungal cells with a first construct and a second construct, wherein the first construct comprises a first polynucleotide flanked on both sides by nucleotides homologous to a first locus in the genome of the protoplast and the second construct comprises a second polynucleotide flanked on both sides by nucleotides homologous to a second locus in the genome of the protoplast, wherein the transformation results in integration of the first construct into the first locus and the second construct into the second locus by homologous recombination, wherein at least the second locus is a first selectable marker gene in the protoplast genome, and wherein the first polynucleotide comprises a mutation and/or a genetic control element;
b.) purifying homokaryotic transformants by performing selection and counter-selection; and
c.) growing the purified transformants in media conducive to regeneration of the filamentous fungal cells.

2. A method for producing a filamentous fungal strain, the method comprising:
a.) transforming a plurality of protoplasts prepared from a culture of filamentous fungal cells with a first construct and a second construct, wherein the first construct comprises a first polynucleotide flanked on its 5′ end by nucleotides homologous to a target locus in the genome of the protoplast and the second construct comprises a second polynucleotide flanked on its 3′ end by nucleotides homologous to the target locus in the genome of the protoplast, wherein the first polynucleotide on its 3′ end and the second polynucleotide on its 5′ end comprise overlapping complementary portions of a selectable marker gene, and wherein the first construct and/or the second construct further comprise a mutation or genetic control element, wherein the transformation results in integration of the first and the second polynucleotide and the mutation or the genetic control element into the target locus by homologous recombination, wherein the target locus comprises a target filamentous fungal gene;
b.) purifying homokaryotic transformants by performing selection and counter-selection; and
c.) growing the purified transformants in media conducive to regeneration of the filamentous fungal cells.

3. The method of claim 1, wherein each protoplast from the plurality of protoplasts is transformed with a single first construct from a plurality of first constructs and a single second construct from a plurality of second constructs, wherein the first polynucleotide in each first construct from the plurality of first constructs comprises a different mutation and/or genetic control element; and wherein the second polynucleotide in each second construct from the plurality of second constructs is identical.

4. The method of claim 1, further comprising repeating steps a-c to generate a library of filamentous fungal cells, wherein each filamentous fungal cell in the library comprises a first polynucleotide with a different mutation and/or genetic control element.

5. The method of claim 1, wherein the first polynucleotide encodes a target filamentous fungal gene or a heterologous gene.

6. The method of claim 1, wherein the mutation is a single nucleotide polymorphism.

7. The method of claim 1, wherein the genetic control is a promoter sequence and/or a terminator sequence.

8. (canceled)

9. The method of claim 1, wherein steps a-c are performed in wells of a microtiter plate and/or are automated.

10.-11. (canceled)

12. The method of claim 1, wherein the filamentous fungal cells are Aspergillus niger or teleomorphs or anamorphs thereof.

13. The method of claim 1, wherein the filamentous fungal cells possess a non-mycelium forming phenotype.

14. The method of claim 1, wherein the fungal cell possesses a non-functional non-homologous end joining (NHEJ) pathway.

15. The method of claim 14, wherein the NHEJ pathway is made non-functional by exposing the cell to a chemical inhibitor.

16. The method of claim 5, wherein the first locus is for the target filamentous fungal gene or a second selectable marker gene in the protoplast genome.

17. (canceled)

18. The method of claim 16, wherein the first selectable marker gene, the second selectable marker gene or the second polynucleotide is selected from an auxotrophic marker gene, a colorimetric marker gene or a directional marker gene.

19.-36. (canceled)

37. A method for preparing filamentous fungal cells for storage, the method comprising:
preparing protoplasts from a fungal culture comprising filamentous fungal cells, wherein the preparing the protoplasts comprises removing cell walls from the filamentous fungal cells in the fungal culture;
isolating the protoplasts; and
resuspending the isolated protoplasts in a mixture comprising dimethyl sulfoxide (DMSO) at a final concentration of 7% v/v or less.

38.-43. (canceled)

44. The method of claim 37, further comprising adding 40% v/v polyethylene glycol (PEG) to the mixture comprising DMSO prior to storing the protoplasts, wherein the PEG is added to a final concentration of 8% v/v or less.

45. (canceled)

46. The method of claim 37, further comprising distributing the protoplasts into microtiter plates prior to storing the protoplasts.

47.-48. (canceled)

49. The method of claim 37, wherein the filamentous fungal cells in the fungal culture are Aspergillus niger or teleomorphs or anamorphs thereof.

50. A system for generating a fungal production strain, the system comprising:
one or more processors; and
one or more memories operatively coupled to at least one of the one or more processors and having instructions stored thereon that, when executed by at least one of the one or more processors, cause the system to perform the method of claim 1.

51.-81. (canceled)

82. The method of claim 2, wherein each protoplast from the plurality of protoplasts is transformed with a single first construct from a plurality of first constructs and a single second construct from a plurality of second constructs.

83. The method of claim 2, further comprising repeating steps a-c to generate a library of filamentous fungal cells, wherein each filamentous fungal cell in the library comprises a different mutation and/or genetic control element in the target locus.

84. The method of claim 2, wherein the mutation is a single nucleotide polymorphism.

85. The method of claim 2, wherein the genetic control element is a promoter sequence and/or a terminator sequence.

86. The method of claim 2, wherein steps a-c are performed in wells of a microtiter plate and/or are automated.

87. The method of claim 2, wherein the filamentous fungal cells are Aspergillus niger or teleomorphs or anamorphs thereof.

88. The method of claim 2, wherein the filamentous fungal cells possess a non-mycelium forming phenotype.

89. The method of claim 2, wherein the fungal cell possesses a non-functional, non-homologous end joining (NHEJ) pathway.

90. The method of claim 89, wherein the NHEJ pathway is made non-functional by exposing the cell to a chemical inhibitor.

91. The method of claim 2, wherein the selectable marker gene is selected from an auxotrophic marker gene, a colorimetric marker gene or a directional marker gene.

92. A system for generating a fungal production strain, the system comprising:
one or more processors; and
one or more memories operatively coupled to at least one of the one or more processors and having instructions stored thereon that, when executed by at least one of the one or more processors, cause the system to perform the method of claim 2.
